(19) United States
(12) Patent Application Publication
(10) Pub. No.: US 2026/0160682 A1
(43) Pub. Date: Jun. 11, 2026

(54) METHOD FOR DETECTING CHEMICAL MESSENGER USING SSDNA FUNCTIONALIZED SENSOR AND RELATED METHOD FOR MAKING THE SSDNA FUNCTIONALIZED

(71) Applicant: UNIVERSITY OF CENTRAL FLORIDA RESEARCH FOUNDATION, INC., ORLANDO, FL (US)

(72) Inventors: DEBASHIS CHANDA, OVIEDO, FL (US); ARITRA BISWAS, ORLANDO, FL (US)

(21) Appl. No.: 19/395,009

(22) Filed: Nov. 20, 2025

Related U.S. Application Data
(63) Continuation-in-part of application No. 17/661,141, filed on Apr. 28, 2022.
(60) Provisional application No. 63/202,894, filed on Jun. 29, 2021.

(51) Int. Cl. G01N 21/552; G01N 27/327; G01N 33/49; G01N 33/543; G01N 33/569

(52) U.S. Cl. CPC G01N 21/554; G01N 27/3276; G01N 33/49; G01N 33/5438; G01N 33/569

(57) ABSTRACT

A method is for detecting a chemical messenger within a sample of blood. The method may include flowing the sample of blood over a sensing surface of a plasmonic array biosensor. The sensing surface of the plasmonic array biosensor may have an ssDNA aptamer against the chemical messenger. The method may further include binding the chemical messenger in the sample of blood to the ssDNA aptamer of the plasmonic array biosensor, and detecting the chemical messenger in the sample of blood based upon LSPR altering a reflected optical signal from the plasmonic array biosensor.

### RELATED APPLICATION

This application is continuation-in-part of application Ser. No. 17/661,141 filed Apr. 28, 2022, which is based upon prior filed copending Application No. 63/202,894 filed Jun. 29, 2021. This application also claims priority to copending Application No. 63/747,194 filed Jan. 20, 2025. The entire subject matter of these applications is incorporated herein by reference in its entirety.

### GOVERNMENT RIGHTS

This invention was made with government support under grant ECCS-1808045 awarded by the National Science Foundation. The government has certain rights in the invention.

### TECHNICAL FIELD

The present disclosure relates to the field of biosensors, and, more particularly, to a biosensor for detecting a virus and related methods.

### BACKGROUND

Viral infections are one of the topmost health concerns worldwide, especially those highly contagious that emerge from isolated geographical clusters and evolve rapidly into pandemics. For instance, the Dengue virus (DENV), a member of the Flaviviridae family transmitted by female mosquitoes (Aedes aegypti or Aedes albopictus), produces a spectrum of clinical illnesses ranging from acute Dengue fever (DF), to deadly hemorrhagic fever (DHF) or the Dengue shock syndrome (DSS). The four DENV serotypes (DENV1, DENV2, DENV3, and DENV4) are a prevailing threat to almost half of the world's population, especially those at tropical latitudes where viral vectors are abundant. In fact, it is estimated that 50 million DENV infections occur around the world each year and approximately 1% of these infections will require hospitalization as they develop into DHF or DSS. Currently, there is no specific treatment for DENV infections, partly because the pathogenesis is not clearly understood. Furthermore, efforts to develop and deploy an effective vaccine against all DENV serotypes has remained elusive, and the current approved vaccine (CYT-TVDV, Sanofi Pasteur) still has below 60% efficacy against the four DENV serotypes in children 2-17 years old. Thus, the early detection followed by aggressive clinical intervention remains as one of the best ways to manage the infection and prevent chronic pathologies or death, and at the same time it becomes an strategic measure to contain the spread.

### SUMMARY

Generally, a method is for detecting a chemical messenger within a sample of blood. The method comprises processing the sample of blood with a microfluidic blood plasma separator and a plasmonic array biosensor, and flowing the sample of blood over a sensing surface of the plasmonic array biosensor. The sensing surface of the plasmonic array biosensor is functionalized with a single stranded DNA (ssDNA) aptamer against the chemical messenger. The method also includes binding the chemical messenger in the sample of blood to the ssDNA aptamer of the plasmonic array biosensor, and detecting the chemical messenger in the sample of blood based upon a localized surface plasmon resonance (LSPR) shift altering a reflected optical signal from the plasmonic array biosensor.

In particular, the detecting comprises shining an optical signal into the plasmonic array biosensor and detecting the LSPR shift of the reflected optical signal. The chemical messenger may comprise at least one of dopamine, serotonin, and epinephrine, for example.

Also, the method may also include flowing a buffer solution and the sample of blood through the microfluidic blood plasma separator until the reflected optical signal stabilizes. The processing may comprise receiving the sample of blood and the buffer solution through separate inlets. The method may also include increasing a flow of the sample of blood until plasma separation occurs to provide a plasma sample from the sample of blood, and performing the detecting on the plasma sample. The method may further include incubating the plasma sample from the blood sample, passing the plasma over the sensing surface of the plasmonic array biosensor, and subsequently to the passing, flushing the microfluidic blood plasma separator with the buffer solution.

In some embodiments, the method may also include reducing nonselective binding from proteins in the sample of blood based upon a passivation layer on the plasmonic array biosensor. The detecting of the chemical messenger in the sample of blood may be performed detecting of the chemical messenger in the sample of blood at a concentration less than 0.2 μg/mL, and in less than 6 minutes.

Another aspect is directed to a method for making a biosensor for detecting a chemical messenger within a sample of blood. The method comprises positioning a microfluidic blood plasma separator on a substrate of a plasmonic array biosensor to process the sample of blood, and functionalizing the plasmonic array biosensor with a ssDNA aptamer against the chemical messenger. A sensing surface of the plasmonic array biosensor is to bind to the chemical messenger in the sample of blood. The plasmonic array biosensor is to detect the chemical messenger in the sample of blood based upon a shift in a LSPR signal, resulting in the LSPR shift in incident probing optical signal reflection.

The method may also include passivating the sensing surface of the plasmonic array biosensor by at least forming a self-assembled monolayer. For example, the self-assembled monolayer may comprise a thiol-terminated 6-mercaptohexanol self-assembled monolayer. In some embodiments, the microfluidic blood plasma separator may be removably clamped onto the substrate. The plasmonic array biosensor may comprise a hole-disc array. For instance, the hole-disc array may have period of 0.5-0.6 μm, a diameter of 0.1-0.3 μm, and a relief depth of 0.2-0.4 μm. Also, the method may include forming a metallic back reflector on the plasmonic array biosensor, forming a dielectric polymer base for the plasmonic array biosensor, and forming a waterproof membrane on the dielectric polymer base.

### BRIEF DESCRIPTION OF THE DRAWINGS

FIG. 1A is a schematic perspective view of an integrated biosensor, according to the present disclosure.

FIG. 1B is an enlarged schematic perspective view of the biosensor of FIG. 1A.

FIG. 1C is a schematic diagram of a dengue virus genome.

FIGS. 1D and 1E are scanning electron microscope top and cross-section images, respectively, of the biosensor of FIG. 1A.

FIG. 1F is a diagram of the reflectance spectrum obtained experimentally and in a numerical simulation, according to the present disclosure.

FIG. 1G is a schematic diagram of the biosensing workflow, according to the present disclosure.

FIG. 2A is a schematic diagram of the biomarker being detected, according to the present disclosure.

FIG. 2B is a diagram of bovine serum albumin (BSA) adsorption on a sensor with and without surface passivation, according to the present disclosure.

FIG. 2C is a diagram of aptamer concentration for functionalizing the sensor's surface, according to the present disclosure.

FIG. 2D is a diagram of LSPR time evolution for three DENV2-NS1 protein concentrations in phosphate buffer saline (PBS) and one control, according to the present disclosure.

FIGS. 2E and 2F are diagrams of LSPR shift quantization in PBS and in PBS+BSA, respectively, according to the present disclosure.

FIG. 3 is a diagram of detection of DENV1 and DENV2 NS1 proteins in PBS+BSA and a control (PBS+BSA), according to the present disclosure.

FIG. 4A is a microscope image of blood plasma separation using the integrated microfluidic device, according to the present disclosure.

FIG. 4B is a diagram of biosensing from blood, according to the present disclosure.

FIG. 4C is a diagram of DENV2-NS1 protein detection at different concentrations in blood and one control, according to the present disclosure.

FIG. 5 is a flowchart of a method for detecting a virus in a sample of blood, according to the present disclosure.

FIG. 6 is a flowchart of a method for making a plasmonic array biosensor for detecting a biomarker, according to the present disclosure.

FIG. 7A is a perspective view of integrated plasmonic biosensor platform, according to the present disclosure.

FIGS. 7B-7C are scanning electron microscope (SEM) images of the biosensor showing surface and cross-section views, respectively.

FIGS. 7D-7E are diagrams of finite-difference time-domain (FDTD) predicted local electric near-field enhancement at λ=842 nm, 2 nm above the surface and cross-section, respectively.

FIG. 8A is a representation of the neurotransmitter detection scheme: (i) The sensors are batch-prepared and cleaned, (ii) The sensor surface is functionalized with a thiol-based dopamine specific DNA-aptamer to create a monolayer, (iii) the surface is further passivated with 6-mercaptohexanol (6-MCH) to reduce further biofouling from interfering species, (iv) a buffer matrix containing different neurotransmitters and biomolecules is introduced for selective dopamine binding to the sensor surface.

FIGS. 8B-8C are images of first-order mechanical phase (M1P) showing the surface morphology before and after the binding events, respectively.

FIG. 8D is a diagram of experimental spectra of one of the plasmonic biosensor surface before and after the surface modification events.

FIG. 8E is a diagram of a bridge-plot showing the spectral change for corresponding surface modification events denoted in the x-coordinate.

FIGS. 9A-9B are diagrams of spectral characterization of the sensor responses for different concentrations of dopamine in PBS buffer, for surface functionalization with 57-base pair DNA aptamer and 44-base pair DNA aptamer, respectively.

FIGS. 10A-10D are diagrams of LSPR shift readings vs concentration of dopamine in PBS, spectral characterization of sensor response in the presence of interfering species (L-DOPA, Epinephrine, Homo-vanillic acid) compared to that of dopamine for the same concentration (10 mM) in PBS-buffer, LSPR Shift vs concentration of dopamine in BSA-spiked PBS solution, and in artificial cerebrospinal fluid (1× aCSF), respectively.

FIG. 11A is a schematic top plan view of a microfluidics integrated biosensor, according to the present disclosure.

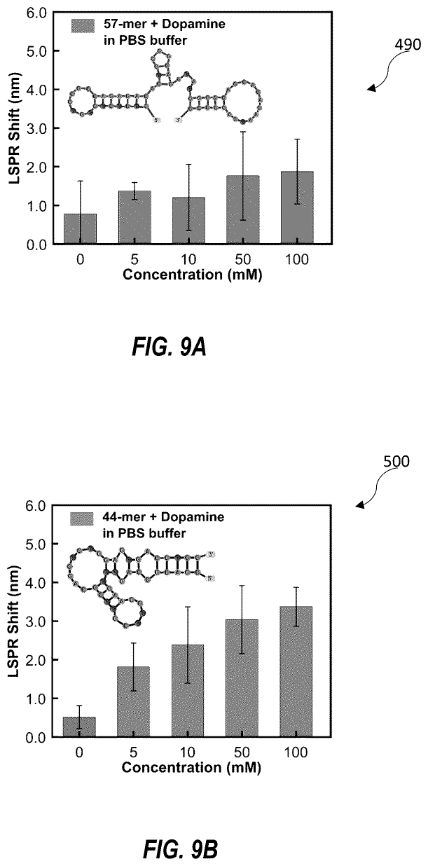

FIGS. 9A-9B are diagrams of spectral characterization of the sensor responses for different concentrations of dopamine in PBS buffer, for surface functionalization with 57-base pair DNA aptamer and 44-base pair DNA aptamer, respectively.

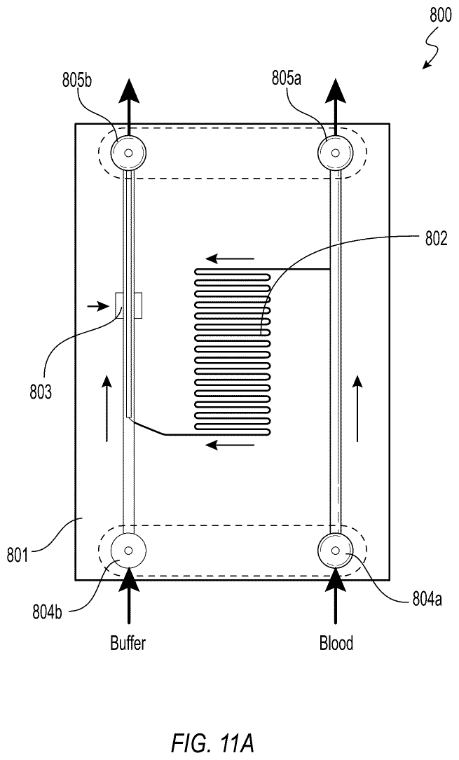

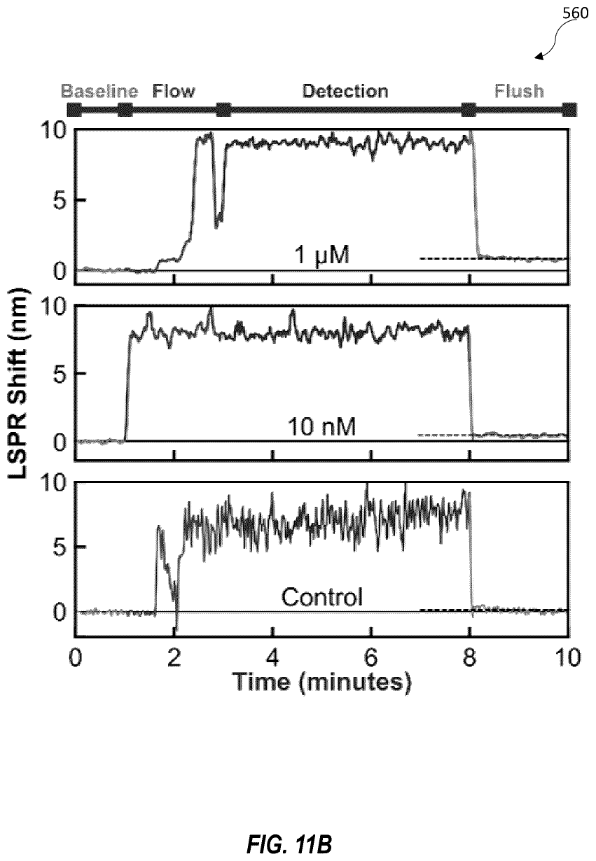

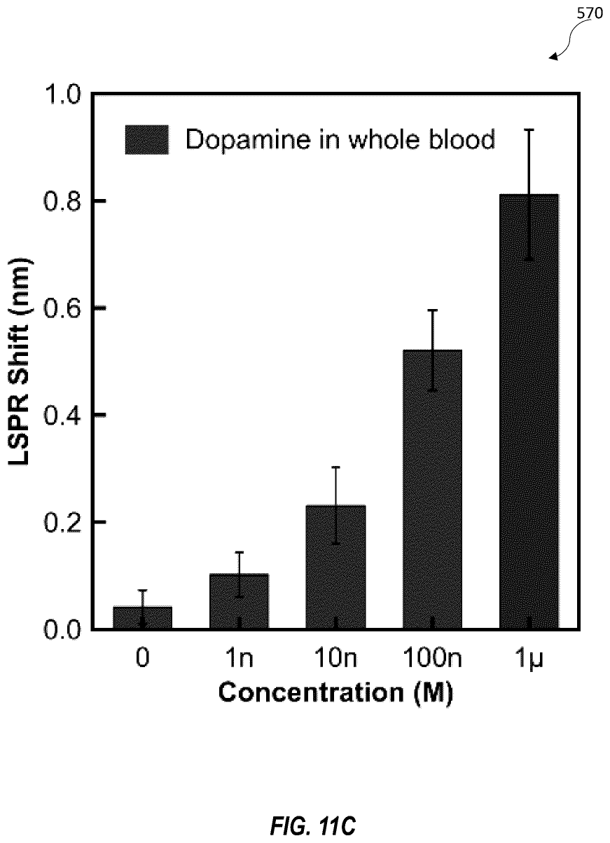

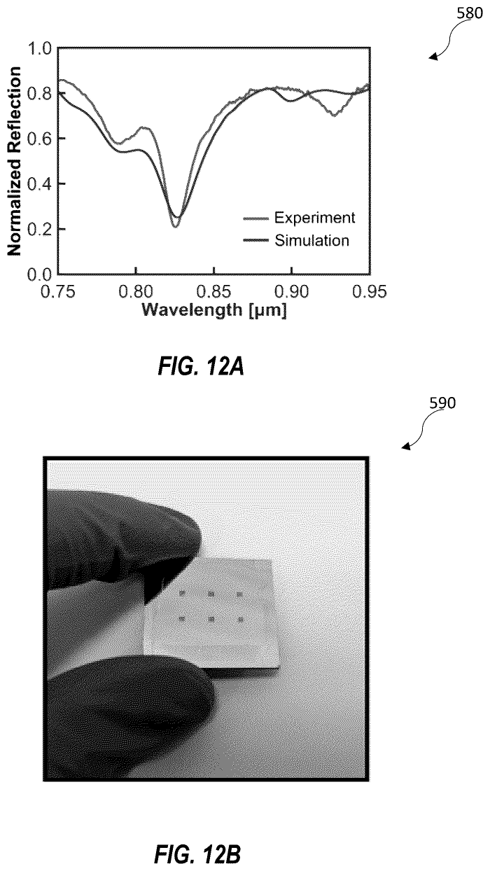

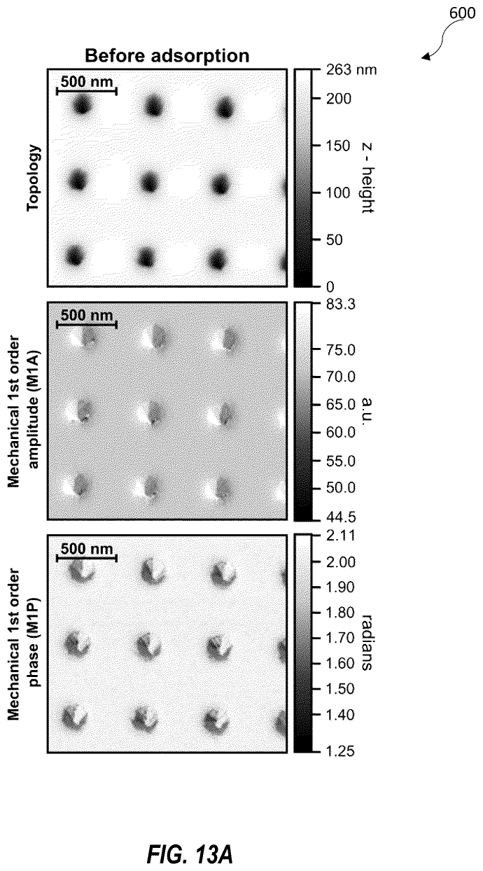

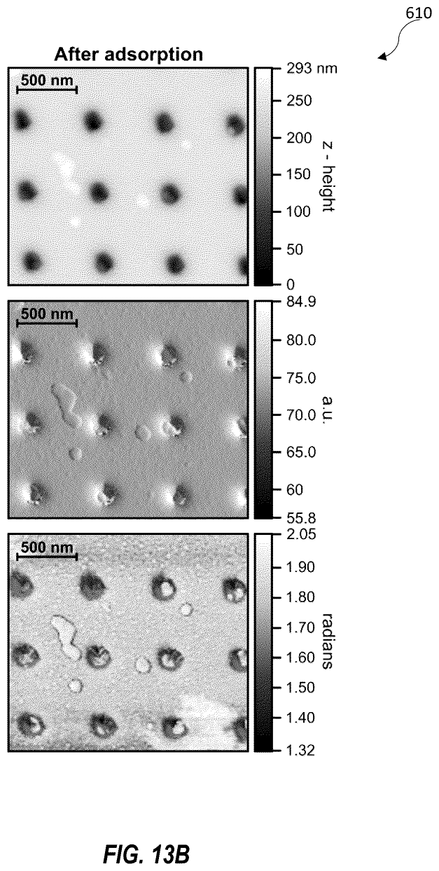

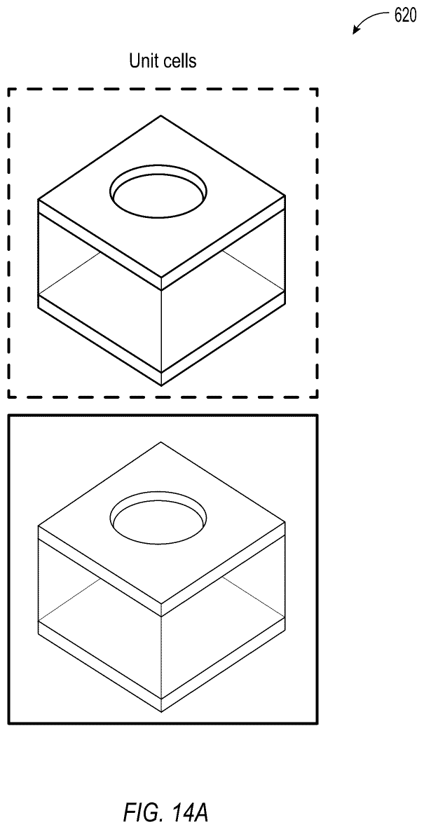

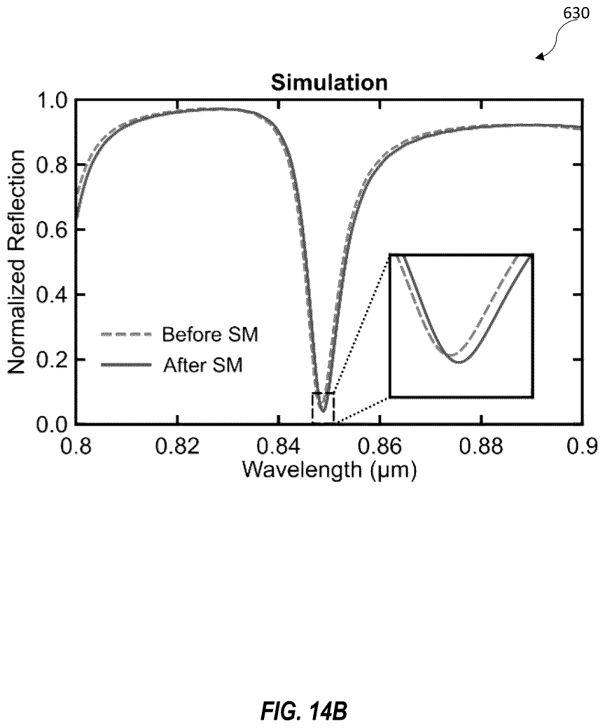

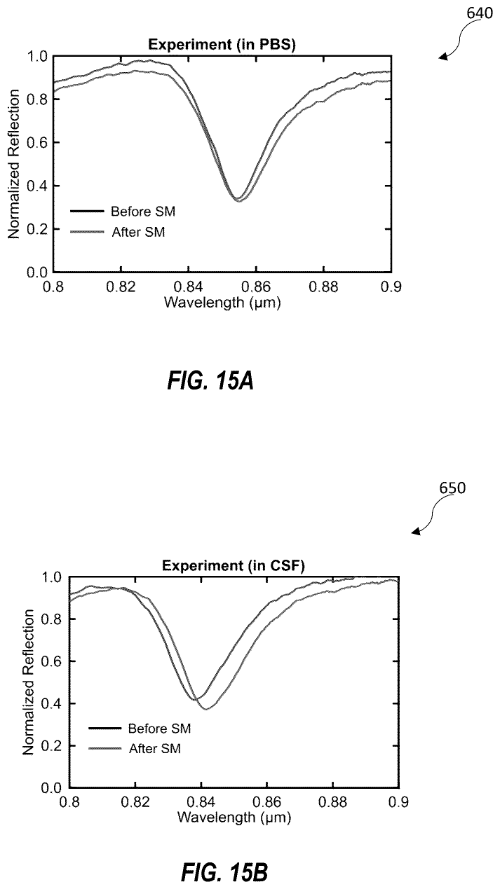

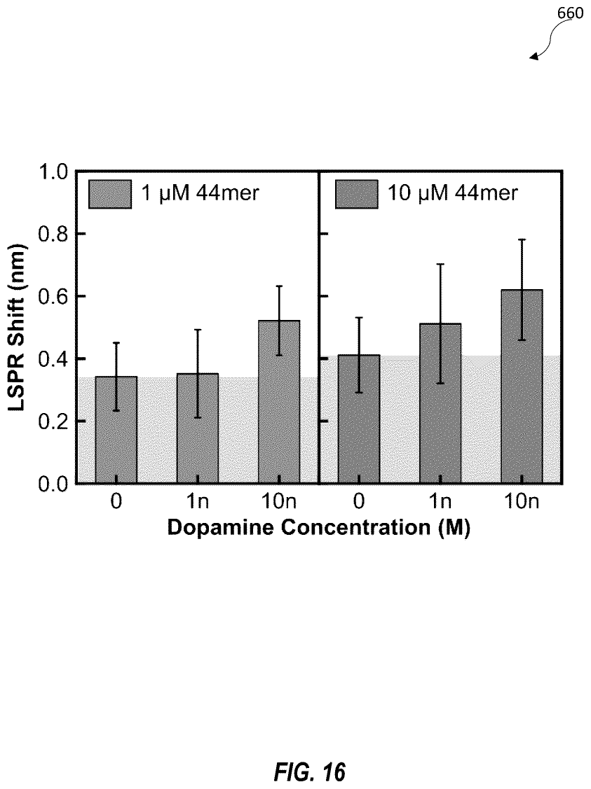

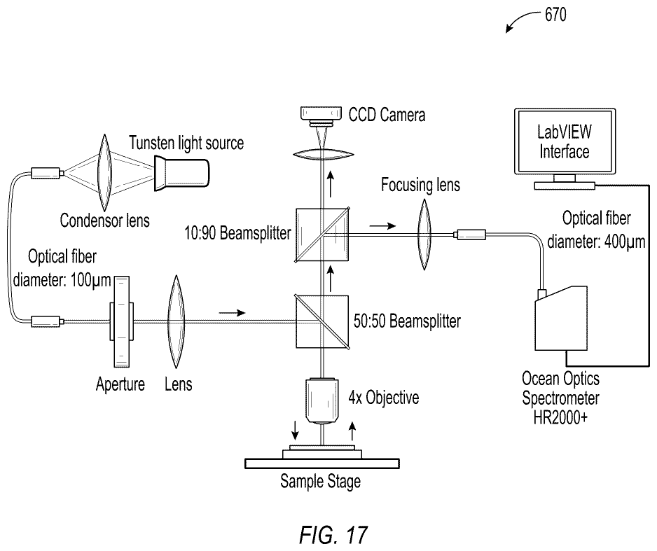

FIGS. 11B-11C are diagrams of time-dynamics of the LSPR-shift signal at different stages during measurement, and LSPR Shift versus concentration of dopamine spiked in bovine whole blood, respectively.

FIG. 12A is a diagram of a comparison of experimental and simulated reflectance response of the plasmonic biosensor, according to the present disclosure.

FIG. 12B is an image of a plasmonic biosensor, according to the present disclosure.

FIGS. 13A-13B are diagrams of before and after aptamer functionalization, showing surface topography (top row), mechanical 1st order amplitude (middle row), and mechanical 1st order phase (bottom row), respectively.

FIG. 14A is a unit cell representation of plasmonic biosensor before (green box) and after (red box) surface modification, according to the present disclosure.

FIG. 14B is a diagram of simulated normalized reflection LSPR spectra of the plasmonic sensor before and after surface modification, according to the present disclosure.

FIGS. 15A-15B are diagrams of measured raw spectral reflection data for two cases: in PBS, and in CSF, before and after surface modification, respectively.

FIG. 16 is a diagram of a comparison of aptameric concentration response with dopamine concentration in PBS for 1 μM (left) and 10 μM (right) aptameric concentration on the biosensor surface, according to the present disclosure.

FIG. 17 is a schematic diagram of an example setup, according to the present disclosure.

### DETAILED DESCRIPTION

The present disclosure will now be described more fully hereinafter with reference to the accompanying drawings, in which several embodiments of the invention are shown. This present disclosure may, however, be embodied in many different forms and should not be construed as limited to the embodiments set forth herein. Rather, these embodiments are provided so that this disclosure will be thorough and complete, and will fully convey the scope of the present disclosure to those skilled in the art. Like numbers refer to like elements throughout, and base 100 reference numerals are used to indicate similar elements in alternative embodiments.

The rapid spread of viral infections demands early detection strategies to minimize proliferation of the disease. Existing polymerase chain reaction (PCR) based viral detection techniques are slow and tedious, which poses technological limitations. Here, a plasmonic biosensor to detect Dengue virus is disclosed, which was chosen as a model virus, via its nonstructural protein NS1 biomarker. The sensor, which is functionalized with a synthetic single-stranded DNA oligonucleotide, provides high affinity towards the NS1 protein present in the virus genome. The present disclosure provides for the detection of the NS1 protein at a concentration of 0.1-0 μg/mL in bovine blood using an on-chip microfluidic plasma separator integrated with the plasmonic sensor, which covers the clinical threshold of 0.6 μg/mL of high risk of developing Dengue hemorrhagic fever. The present disclosure may demonstrate potential application of these microfluidic optical biosensors for early detection of wide range of viral infections, and may provide a rapid clinical diagnosis of infectious diseases directly from minimally processed biological samples at a point of care location.

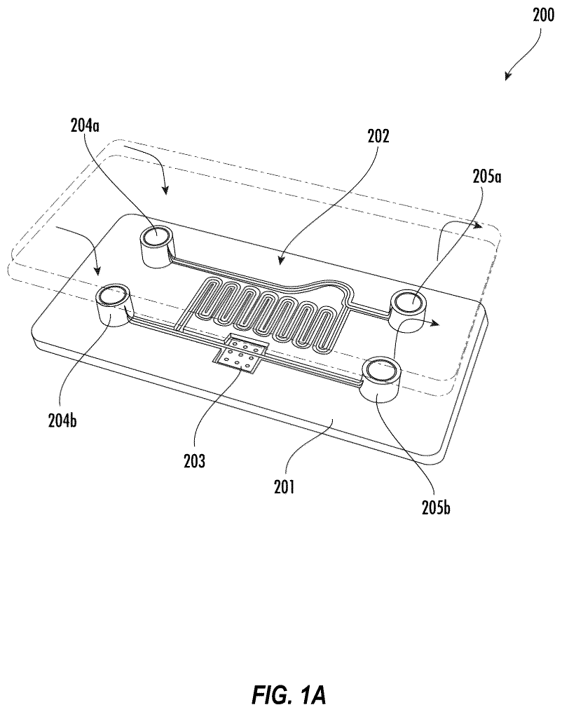

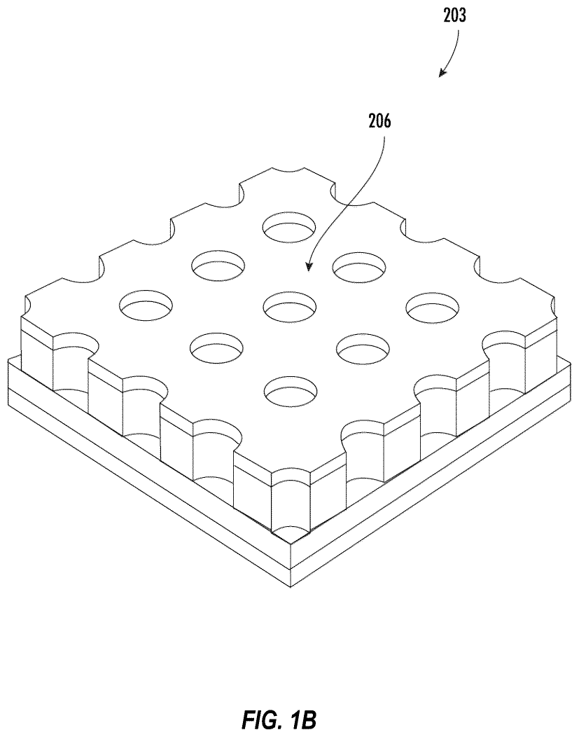

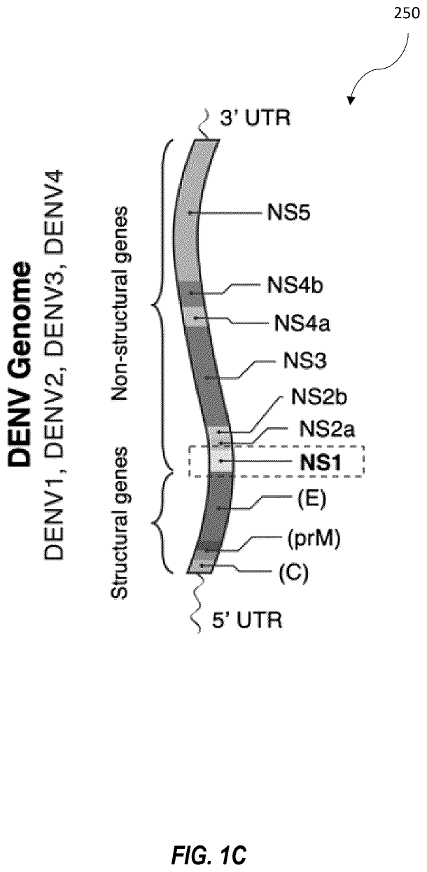

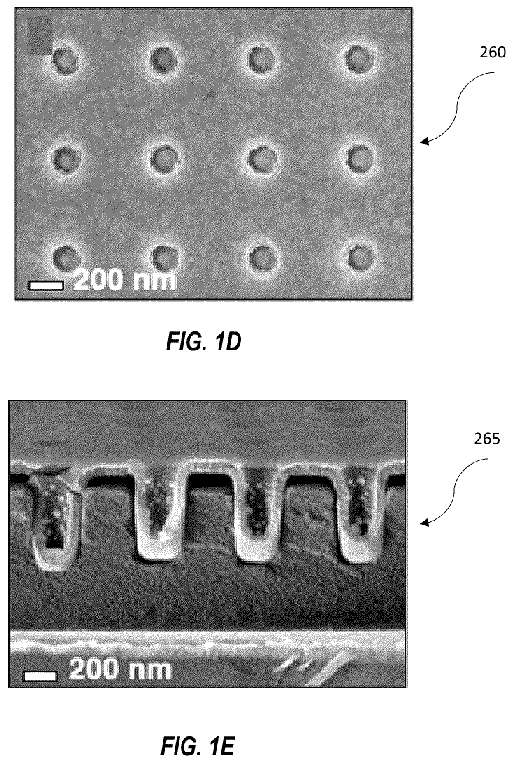

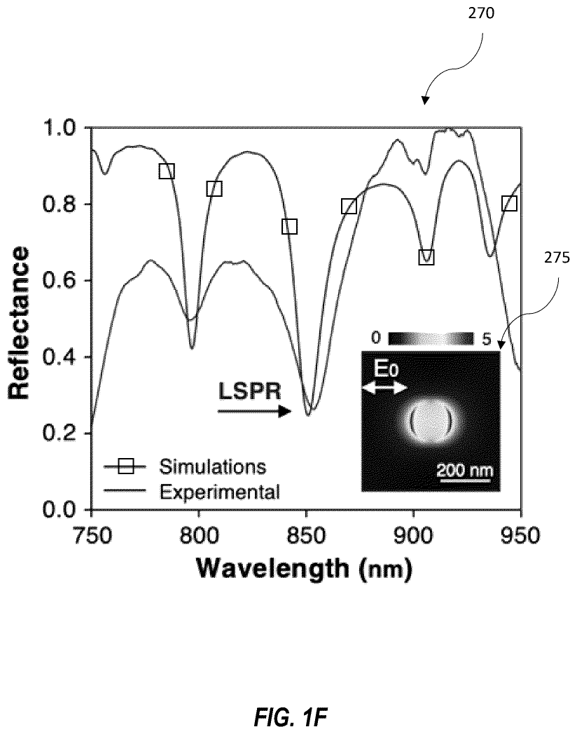

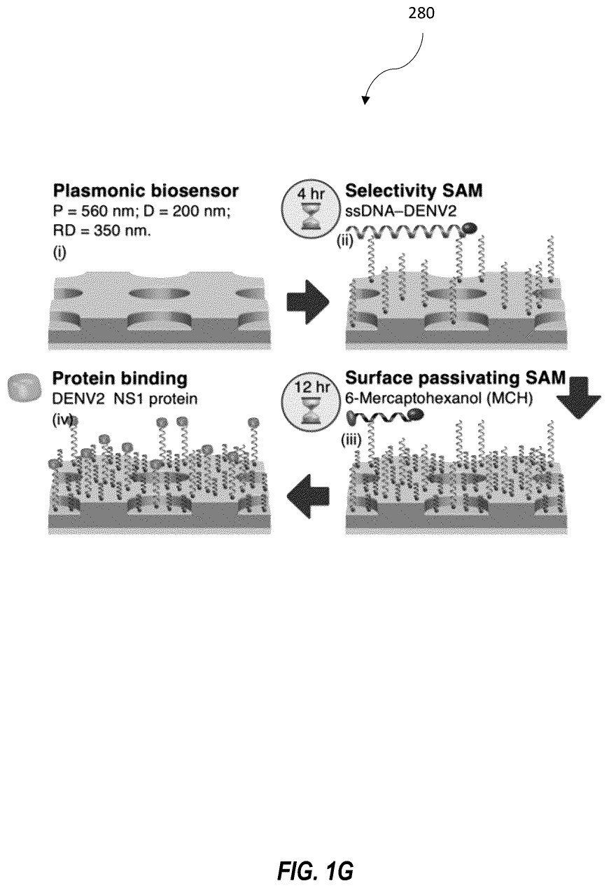

FIG. 1A: Integrated dengue virus biosensor comprises an in-line microfluidic blood plasma separator, and a cavity-coupled nanoimprinted plasmonic array. FIG. 1B shows the nanoimprinted plasmonic biosensor comprising a gold mirror (80 nm thick), a dielectric film spacer (˜700 nm thick) embossed with a square array of holes, a conformal thin film of aluminum oxide layer (20 nm thick) as fluid barrier, and a thin film gold (30 nm thick). FIG. 1C shows the Dengue virus genome shared among the four DENV serotypes and comprising three structural (C, prM, and E) and seven nonstructural (NS) proteins, including NS1. FIGS. 1D-1E are scanning electron microscope images, top view (FIG. 1D), and cross section (FIG. 1E), of one representative nanoimprinted sensor. FIG. 1F shows a reflectance spectra obtained using FDTD simulations (square marked curve) and experimentally (solid curve). The inset of FIG. 1F represents the electric field enhancement obtained 5 nm above the circular hole at the LSPR. FIG. 1G shows a diagram 280 of biosensing workflow. (i) The sensors are batch prepared and properly cleaned prior to functionalization. (ii) Thiol-terminated ssDNA aptamer specific to nonstructural DENV2-NS1 protein binds covalently to the gold surface. This step requires incubation for 4 hours. (iii) Thiol-terminated 6-mercaptohexanol (MCH) self-assembly monolayer binds covalently to the empty surface area to block nonspecific binding and decrease protein fouling present in biological fluids. This process requires incubation for 12 hours. (iv) DENV2-NS1 protein detection is performed via aptamer binding, which produces a readable LSPR shift.

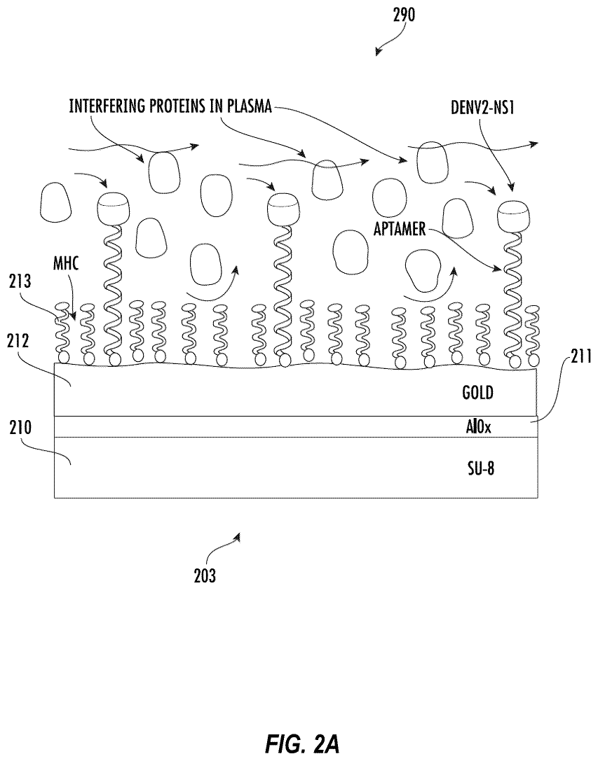

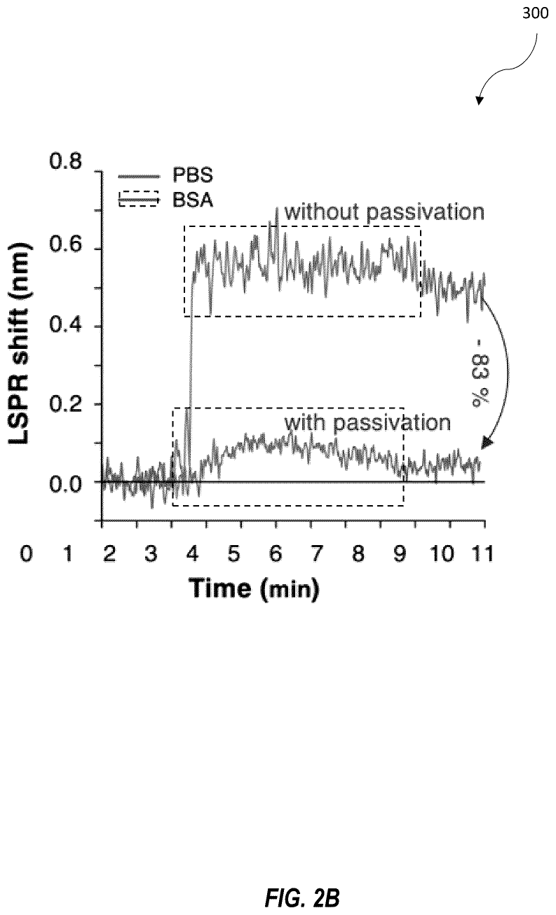

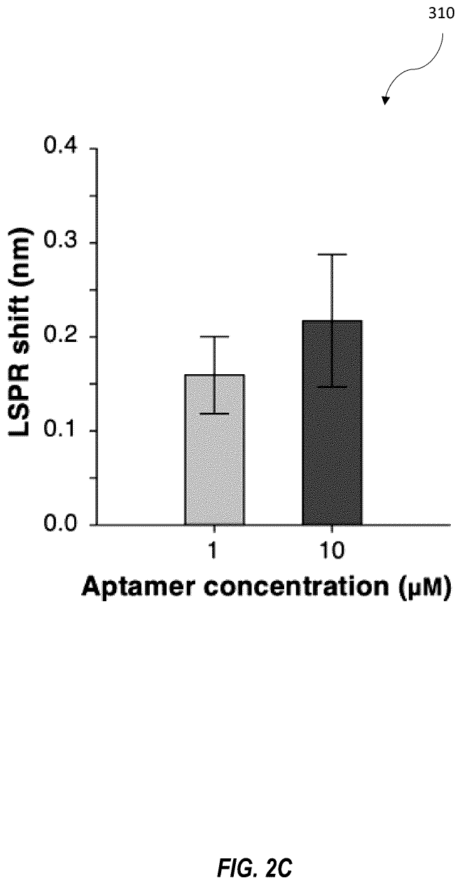

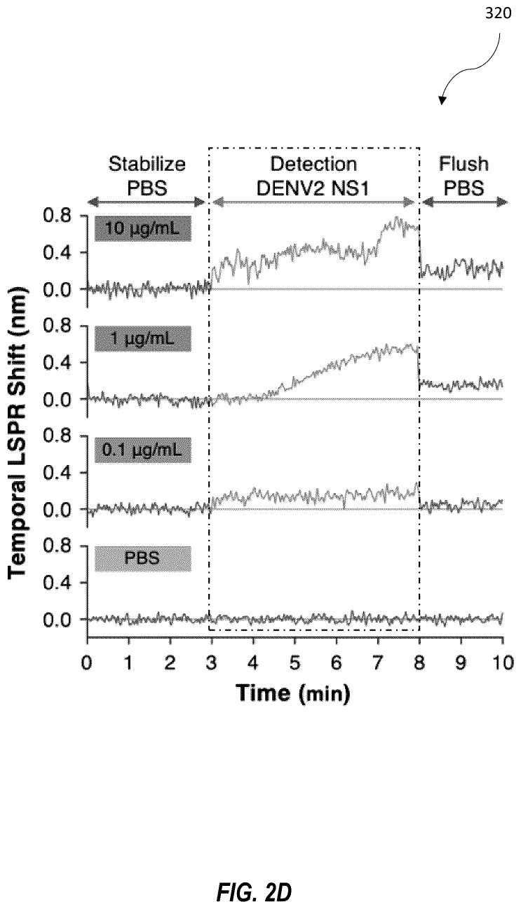

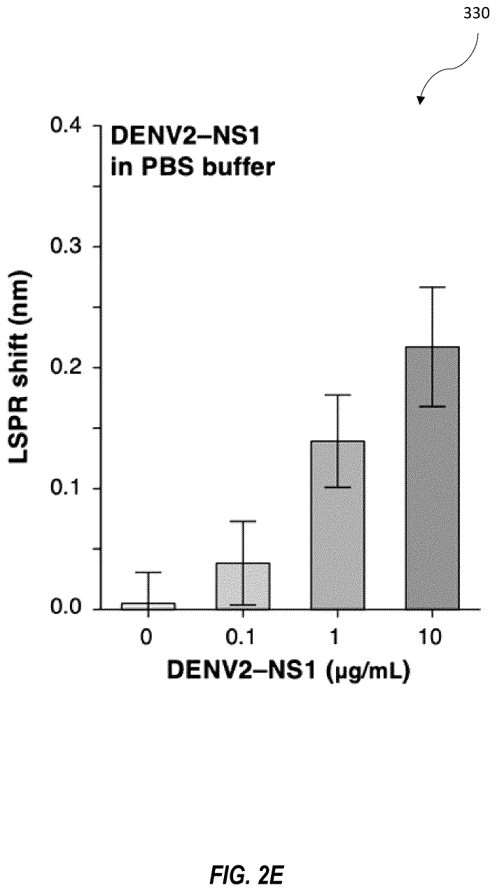

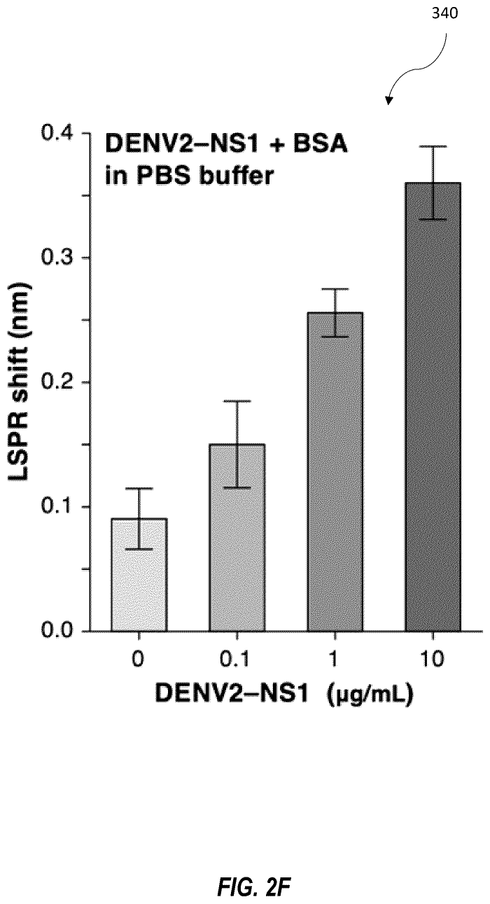

FIG. 2A shows a diagram 290 of biosensing from biological fluid. The surface passivation strategy decreases protein fouling while permitting the binding of the biomarker to the ssDNA aptamer. FIG. 2B shows a diagram 300 of thiol-terminated MCH surface passivation against BSA adsorption (100 μg/mLin PBS). Surface passivated sensor reduces up to 83% the sensor residual response with respect to its non-passivated counterpart. FIG. 2C shows a diagram 310 of biosensing with simultaneous passivation and functionalization. The sensors are functionalized with two aptamer concentrations (1 μM and 10 μM) and tested with same 10 μg/mL DENV2-NS1 concentration in PBS. FIG. 2D shows a diagram 320 of LSPR time evolution for three DENV2-NS1 protein concentrations (i.e., 0.1, 1, and 10 μg/mL) in PBS and one control (only PBS, or zero concentration). The sensorgrams show the time stamps at which the flow events were happening via the dashed box. The dashed box marks a detection period; before the dashed box marks the stabilizing period; and after the dashed box marks the flush period. FIG. 2E shows a diagram 330 for LSPR shift quantization of the four sensorgrams in FIG. 2D as a function of DENV2-NS1 protein concentration. FIG. 2F shows a diagram 340 of LSPR shift quantization of three DENV2-NS1 protein concentration in PBS solution with additional 100 μg/mL BSA concentration, and control sample (only PBS+BSA, or zero concentration). Error bars are the standard deviation from the mean.

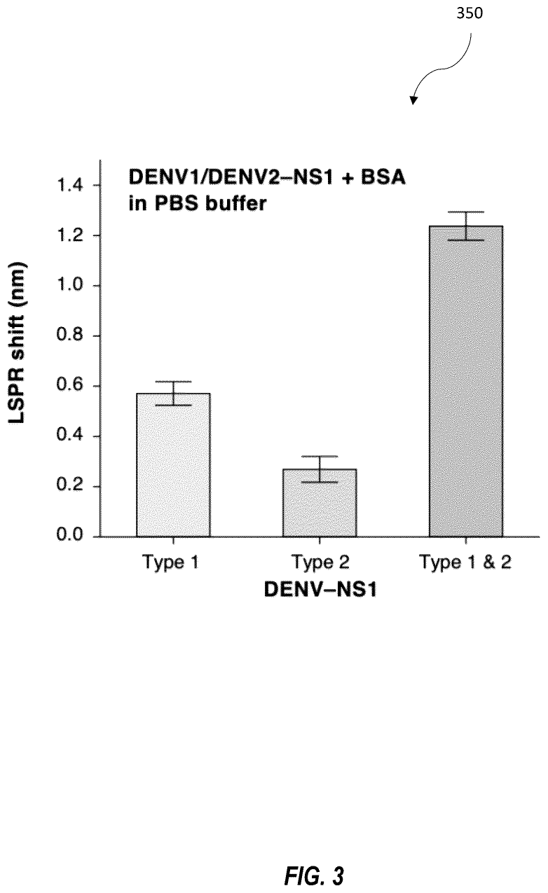

FIG. 3 shows a diagram 350 of detection of DENV1 and DENV2 NS1 proteins in PBS. The close genetic match of the NS1 protein between the DENV1 and DENV2 (71.67%) leads to finite LSPR shift when testing DENV1 and DENV2 separate or DENV1+DENV2 combined. Error bars are the standard deviation from the mean.

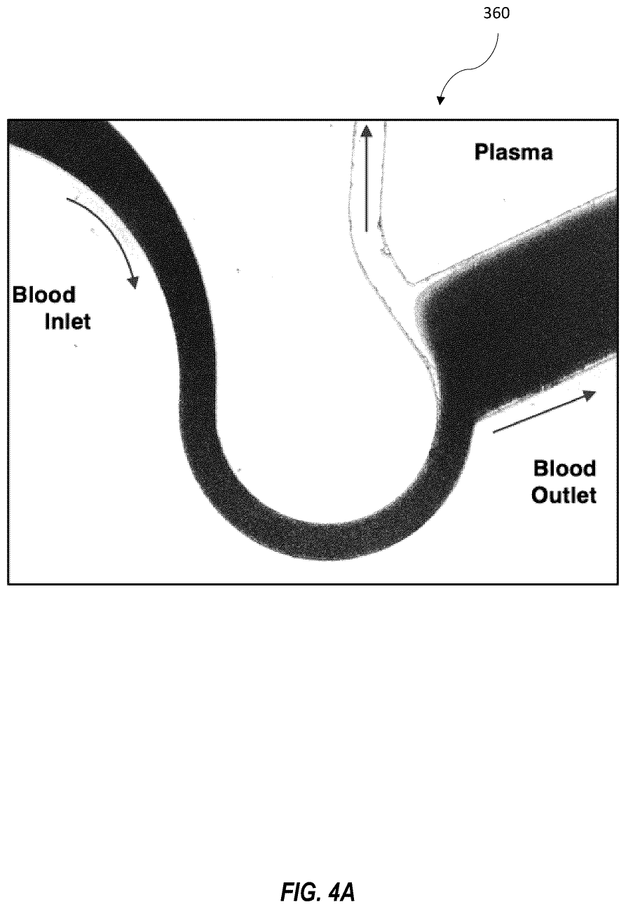

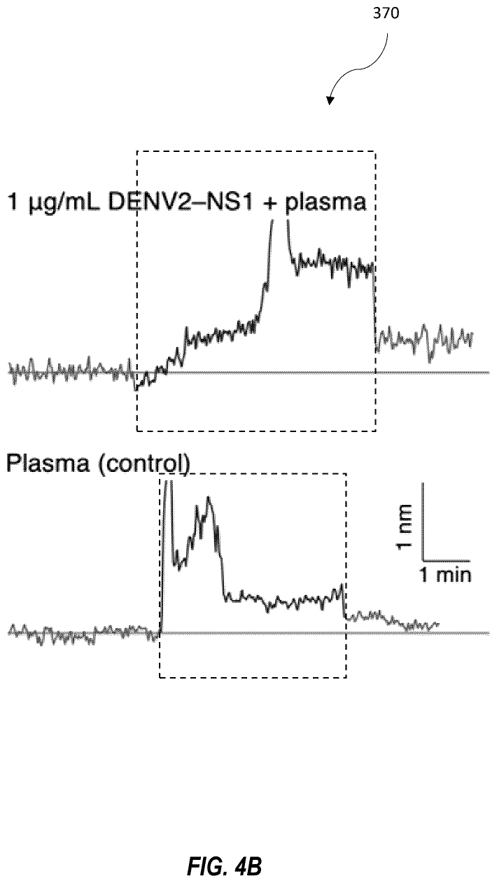

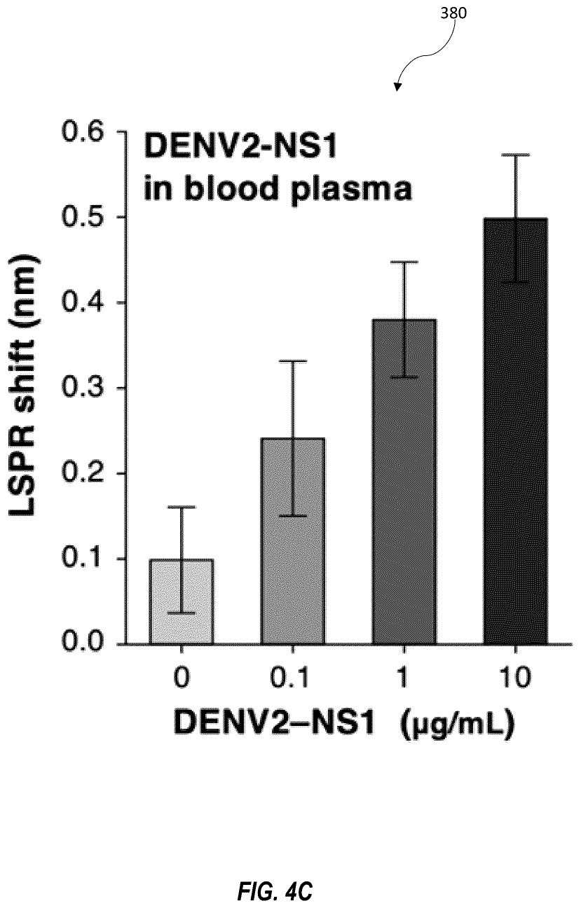

FIG. 4A shows a microscope photograph image 360 of the blood plasma separation during operation. FIG. 4B shows a diagram 370 of two representative sensorgrams during the biosensing demonstration from blood. First a baseline is established by flowing PBS first, then plasma separation is started followed by an incubation time (typ. 3 min) and finalized with PBS flush. FIG. 4C shows a diagram 380 of DENV2-NS1 protein detection at different concentrations (0.1, 1, and 10 μg/mL) in blood and one control (just blood). This biosensing strategy demonstrates biomarker detection from complex biological fluids, such as blood plasma. Error bars are the standard deviation from the mean.

Viral infections, such as DENV, are diagnosed using antibody, protein biomarkers, or messenger ribonucleic acid (mRNA) from a biological sample. On one hand, the enzyme-linked immunosorbent assay (ELISA) is the clinical diagnostic gold standard for seral antibodies or proteins detection, which is an indirect approach to identify an active or past viral infection. On the other hand, the reverse transcriptase polymerase chain reaction (RT-PCR) specifically targets viral mRNA strands with high accuracy, which is ideal for the diagnosis of active viral infections with specific genome specificity. However, the cost of a time-consuming process, the dedicated infrastructure, and the extensive sample preparation in both cases restricts the proliferation beyond commercial laboratories and academic environments. Alternative label-free affinity biosensors promise to develop assays for the rapid diagnosis of pathogenetic biomarkers, for example those based on localized surface plasmon (LSP). These devices are extremely sensitive due to the subwavelength electric field confinement produced by the LSP at resonance which makes them ideal for small biomolecule detections. When properly functionalized with an affinity recognition layer, these sensors can detect minute concentrations of analyte down to the picomolar to femtomolar concentrations.

In the present disclosure, a cavity-coupled plasmonic biosensor 200 integrated with a microfluidic blood plasma separator may detect the DENV biomarker directly from blood as schematically is shown in FIG. 1A. The cavity coupling improves the quality factor Q (bandwidth, Δλ) of the LSPR while nanoimprinting based patterning ensures a deterministic resonance (λ_{R}). Both of these attributes were previously shortcomings and prevented the commercial development of plasmonic sensors.

In order to make it target specific, the plasmonic biosensor is functionalized with a ssDNA aptamer against the nonstructural NS1 protein from DENV2 serotype, (diagram 250, FIG. 1C). Aptamers are synthetic short single-stranded nucleic acids which are specifically designed to bind, with high genetic selectivity, a myriad of targets, from ions, small molecules, and proteins to supramolecular complexes. They outperform antibodies, typically used in affinity layers in biosensing, in many different ways. For example, single-stranded DNA (ssDNA) aptamers are chemically synthesized using the systematic evolution of ligands by exponential enrichment (SELEX) method to bind the target with high affinity, and cloned afterwards. This makes the aptamer production process cost-effective for highly reproducible biosensing platforms as no living animal model is needed for the production. In addition, the present disclosure includes a surface passivation scheme based on thiol-terminated MCH self-assembled monolayer to minimize nonselective binding from proteins present in biological fluids, such as blood plasma. With this genetically modified sensor design, the present disclosure discloses the detection of NS1 protein from DENV2 at a concentration of 0.1-10 μg/mL in bovine blood using a plasma separating microfluidic chip integrated with this plasmonic biosensor. This concentration range covers the clinical threshold of 0.6 μg/mL for predicting high risk of developing DHF in DENV infected patients.

### Results: Plasmonic Biosensor

LSP oscillations are supported at the interface between dielectric and subwavelength metallic structures. The LSPR is inherently determined by the geometrical configuration, materials, and the surrounding dielectric permittivity. Any modification to these parameters induces a resonance shift, however without any selectivity. That means two similar biomolecules when attached to the metallic interface impart same resonance shifts, making them indistinguishable. Hence, it is essential to incorporate an affinity layer against the target biomarker to produce a selective LSPR shift upon analyte binding to enable label-free affinity-based plasmonic biosensing.

The plasmonic sensor is formed by hybridizing the LSP with an asymmetric Fabry-Perot cavity resonator. The plasmonic nanostructure, unlike typical one metallic element, is composed of two complementary elements, hole/disk, of period/diameter of 560/200 nm and separated by 350 nm relief depth. The pattern is thermally embossed on UV curable epoxy SU-8 and coated with 30 nm of gold (See FIG. 1B). Further, the complementary hole/disk array is far-field coupled with the photonic cavity formed by an optically thick (100 nm) gold back reflector separated from the plasmonic nanostructure by a thick SU-8 spacer film (typically 700 nm). In applications where the plasmonic device is submerged in solvents, including water, for prolonged periods of times leads to SU-8 swelling, which could add finite cavity thickness variations observed in spurious LSPR shift. In order to prevent this detrimental effect, a conformal 20 nm aluminum oxide membrane is grown on the polymeric nanohole array prior to metal evaporation to prevent water diffusion into the epoxy matrix. FIG. 1B depicts the schematic representation of this system and the corresponding scanning electron microscope (SEM) image of one representative device with cavity thickness of ˜700 nm is shown in images 260, 265 of FIGS. 1C-1D. Cavity-coupled plasmonic systems support hybridized LSP oscillation modes in the top metallic nanostructure that, when coupled with a back metallic reflector, form an asymmetric photonic cavity. Light coupled to this cavity produces Fabry-Perot resonant modes and, when overlapped in space and time with LSP, generates hybrid LSP modes. Such hybrid LSP modes offer robustness against fabrication tolerances due to the weak space-time coupling between the cavity and LSP modes. FIG. 1F shows the reflection spectra obtained using finite difference time domain (FDTD) simulations (i.e., Q factor of 53) compared with that obtained experimentally (i.e., Q factor of 37) when immersed in aqueous solution (n=1.333). At resonance (LSPR˜850 nm), the hole/disk array supports a plasmonic resonance (inset 275 in diagram 270 of FIG. 1F) which is extremely sensitive to changes in the surrounding medium, resulting in a measurable shift in the resonance frequency.

### Target Specific Viral Genome Detection

Nonstructural NS1 glycoprotein forms part of the genetic code of all the Flaviviridae virus family, such as the four serotypes of the DENV, the Japanese encephalitis virus, Yellow fever virus, West Nile virus, tick-borne Encephalitis virus, and Zika virus. FIG. 1C shows the DENV genome, which is not different from the general Flaviviridae virus family. However, such a genomic similarity does not translate to a genetic material homogeneity. For example, only 23% of the protein genome is conserved among these virus genera. In addition, DENV2 shares 71.44% with DENV 1, 71.64% with DENV 3, 68.82% with DENV 4, 55.44% with Zika virus, 51.96% with West Nile virus and 45.46% with Yellow fever virus. NS1 proteins, a constituent of the DENV genome, plays an important role in the virus replication process, and is secreted into the blood stream by the infected cells, thus it becomes a representative biomarker for the identification of the infection. It was demonstrated that high levels in acute-phase serum samples correlated with high risk of developing DHF. Hence, detection directly from the blood, or plasma, is essential in order to avoid fatalities.

To target specific detection, the sensor is functionalized with ssDNA aptamer to detect NS1 protein directly from blood. However, detection from minimally processed and unfiltered biological samples in general is challenging due to the protein adsorption on sensor surface. The high protein content in blood plasma tends to electrostatically accumulate on the negatively charged gold surface producing a spurious biofilm accumulation leading to an uncontrolled background masking the positive binding of the target biomarker. In order to address protein fouling, the present disclosure implements a surface passivation based on thiol-terminated MCH self-assembled monolayer, which is a typical surface passivating strategy for gold-based biosensors. The functionalization and biosensing workflow are graphically represented in diagram 280 of FIG. 1G. The plasmonic substrates were batch nanoimprinted (FIG. 1G-i), functionalized with commercially available thiol-terminated ssDNA aptamer against the NS1 protein from DENV2 (DENV2-NS1) serotype (Base Pair Biotechnologies Inc.) (FIG. 1G-ii), followed by surface MCH passivation (FIG. 1G-iii), and finally NS1 protein binding detection using optical reflection spectroscopy and subsequent quantization by LSPR shift (FIG. 1G-iv). This specialized surface functionalization and passivation enabled detection of antigen even in presence of high protein content in the biological sample as can be seen in diagram 290 of FIG. 2A. The sensor passivation was characterized first using 100 μg/mL BSA in PBS as a model for non-specific interfering protein contained in biological samples.

Two non-functionalized plasmonic substrates were prepared with and without surface passivation. This PBS+BSA solution was flowed using a microfluidic chip placed on top of the plasmonic substrate and secured with an acrylic fixture. First, the PBS flows in the microfluidic channel to bring the sensor to a stable state determining the baseline. Then, the PBS+BSA solution flows for five minutes and then flushed away with PBS to remove poorly adhered BSA on the gold surface. Protein adsorption and selective biding in the subsequent biosensing demonstrations, is gauged using the LSPR shift with respect to the baseline. In the plasmonic substrate without passivation, BSA accumulates rapidly within half a minute and remain constantly independent of the continuous BSA flow, which is a sign of surface saturation. The flow of PBS solution flushes any poorly adhered BSA, but the residual LSPR shift (0.497±0.028 nm) denotes a considerable remaining protein adhesion to the substrate, see diagram 300 of FIG. 2B. In contrast, the BSA minimally affects the surface passivated substrate observed in the slower sensor response and the small residual LSPR shift (0.083±0.018 nm) after PBS flush. This strategy achieves about 83% reduction in the residual LSPR shift. After confirming the surface passivation implementation, the plasmonic biosensor with simultaneous ssDNA aptamer functionalization and surface passivation was tested. Two sensors were functionalized with 1 and 10 μM ssDNA aptamer concentration and 10 μg/mL purified DENV2-NS1 protein in PBS buffer was flowed in the same pattern as described in the previous surface passivation characterization. FIG. 2C includes a diagram 310, which shows the LSPR shift, which confirms the sensor binds the biomarker at two strengths commensurate with the surface coverage of the ssDNA aptamer; 10 μM ssDNA aptamer concentration was employed in all the subsequent biosensing demonstrations.

### Detection in PBS

The first detection demonstration is performed in PBS solution where purified DENV2-NS1 protein was spiked at different known concentrations. The purpose of this characterization is to evaluate the binding capability to detect the target biomarker within the clinically relevant concentration for high risk DHF development (0.6 μg/mL). A batch of sensors were fabricated, functionalized and surface passivated. Using the integrated microfluidics system, each sensor was exposed to different DENV2-NS1 protein concentrations, 0.1, 1 and 10 μg/mL including a control solution (in this case just PBS). The sensors were first stabilized with PBS running buffer, then the PBS+DENV2-NS1 solution flowed for five minutes followed by PBS flush, while continuously collecting the reflection spectra at each second interval using a spectrometer automated with a customized LabVIEW (National Instruments) graphic user interface. Typical time evolution of the LSPR shift can be observed in diagram 320 of FIG. 2D.

The results of this characterization are summarized in diagram 330 of FIG. 2E where the LSPR shift is plotted as a function of DENV2-NS1 protein concentration. The fact that the LSPR shift is technically zero for the control solution validates the effect of the aluminum oxide passivating layer that prevents SU-8 swelling when exposed to aqueous solution for the 10-minute period. In addition, the low LSPR shift for 0.1 μg/mL concentration (0.038±0.035 nm) suggests limit of detection in this range. This demonstration shows a promising performance and paves the path for detection of DENV2-NS1 from real biological samples containing elevated concentration of proteins.

### Detection From PBS+BSA

In order to assess the device performance in artificial sample conditions, i.e., saline solution with high protein content BSA, and the target biomarker, the same experiment was repeated, but 100 μg/mL of BSA was added into the PBS buffer. The sensors were batch fabricated, functionalized and surface passivated. FIG. 2F includes a diagram 340, which summarizes the LSPR shift with respect to DENV2-NS1 protein. It is observed that the control solution, PBS+BSA only, has a small background (0.090±0.024 nm) such as that seen during the surface passivation test, see FIG. 2B. However, the LSPR shift as a function of DENV-NS1 protein concentration varies in much the same way than the case without BSA with sensor response of (0.150±0.035 nm) for the smallest DENV2-NS1 concentration. Such a demonstration confirms that this biosensing strategy has the potential to detect the target biomarker even in environments where other non-specific biomolecule binding, BSA in this case, could mask the response. The ssDNA aptamer used was designed against DENV 2-NS1, but due to its 71.46% overlap with DENV1-NS1 genome the biosensor produced a finite response. FIG. 3 includes a diagram 350 showing the LSPR shift response from three PBS+BSA solutions containing 10 μg/mL of DENV 1-NS1, 10 μg/mL of DENV2-NS1 and a mix of 10 μg/mL of each. Henceforth, it is expected that with further aptamer optimization guided by the diversity of the NS1 genetic code, it would be possible to detect DENV1 and DENV2, along others Flaviviridae with high specificity.

### Direct Detection From the Blood

Ultimately, a biosensor capable of performing biomarker detection from minimally prepared blood samples is the key for the point-of-care clinical diagnostics and other applications that require portability. In order to demonstrate the feasibility of performing DENV2 detection using the circulating NS1 protein biomarker directly from bovine blood, the plasmonic biosensor was integrated with an on-chip microfluidic plasma separator, see FIG. 1A. This microfluidic chip uses a series of biophysical and hydrodynamical effects to generate a cell-free region as the result of cell migration and inertial focusing at the bifurcation of an asymmetric flow resistance paths with ratio of at least 2.5:1, where large cells such as red and white blood cells and platelets are deviated towards the low resistance path and cell-free plasma is collected in on the opposite. The hybrid microfluidic-plasmonic chip was fabricated using standard photolithography and nanoimprinting techniques. In addition, the chip was held secured to the gold surface using a customized acrylic clamp. This way, the microfluidic chip is removed, cleaned, and reused multiple times. The samples were batch fabricated, surface functionalized and passivated following the same method described above. Bovine whole blood (˜35% hematocrit (HCT)) was prepared by spiking a deterministic concentration of DENV2-NS1 protein (0.1, 1, and 10 μg/mL) and control in PBS solution which produced blood samples with ˜22.3% HCT. Blood samples with lower HCT perform better than high hematocrit content blood samples using these type of devices, especially in the low flow regime preventing the non-covalent bonded microfluidic chip from delamination.

Detection directly from blood following this scheme requires four steps. In the first step, PBS, and blood flows at low speed at the same time to fill the channels with solution. Once the reflected optical signal is stabilized, the PBS channel closes meanwhile the blood flow increases until plasma separation occurs within the first two minutes, see microscope photograph image 360 of FIG. 4A. Once cell-free plasma flows on the active sensing region, the blood flow is stopped and kept off for another 3 minutes to incubate the plasma sample. Then, the PBS channels is open to flush the plasma and other proteins off the active region. FIG. 4B shows a diagram 370 of two LSPR chronograms, non-boxed regions are the PBS flow events, and the dashed box region correspond to the plasma separation and detection. Spikes in the LSPR are observed mainly due to event related to remnant blood cells, refractive index gradients, loss of LSPR tracking, or the reflectance spectra flattens, but not biomarker binding. However, flushing with PBS solution allows for the reflectance spectra to recover to the baseline state but with an LSPR shift gained by the binding of the biomarker. As expected, the control sample produces a small background in the same range that in the previous biosensing experiment (0.099±0.024 nm). Nevertheless, the DENV 2-NS1 protein was clearly differentiated at different concentrations, see diagram 380 of FIG. 4C. In addition, similar limit of detection is observed (0.1 μg/mL) still within the clinical threshold for high risk of DHF development (0.6 μg/mL).

Other methods of DENV detection mostly use immunoassay approaches -the use of antibodies as the affinity layer. For example, the detection of DENV E-protein (See FIG. 1C) in buffer, Immunoglobulin M, as the immune system response to DENV infection, in diluted human serum, and NS1 protein in diluted human blood plasma were performed using the surface plasmon resonance (SPR) method, with detection limits in the femtomolar rage for DENV E-protein in buffer. Other optical methods such as luminescence of quantum dots conjugated with gold nanoparticle via ssDNA aptamers detect purified DENV DNA complementary strands at femtomolar concentrations in buffer. Unlike other demonstrations, the present disclosure shows the use of a hybrid microfluidic-plasmonic device as a promising technique for the rapid sample preparation (plasma separation) and antigen detection with comparable detection ranges as other SPR based sensors. The present disclosure represents an attractive approach for the detection of viruses directly from blood, with comparable assay time (<10 min) and clinically relevant limit of detection (0.1 μg/mL).

Success in containing the spread of viral infections relies on the early and accurate detection of biomarkers in biological fluids, ideally in the infection stages prior to the onset of life-threatening symptoms. In addition, as several viral infections manifest as similar symptoms at the early stages, and most of them can develop into chronic pathologies, any misdiagnosis can lead to severe medical complications leading to death. In this context, the present disclosure demonstrated a biosensing platform with potential portability for deploying to the sites of interest, for example, in remote locations lacking the adequate infrastructure for prompt screening. In some embodiments, multiplexing, and hardware integration this platform can become a practical solution to detect a myriad of other biomarkers, not only the different genera from the Flaviviridae virus family but to other relevant pathologies associated to viral infections, such as the current COVID-19 pandemic caused by the SARS-CoV-2 virus.

A method is performed by an automated system in order to control the plasma separation process. A method is to functionalize and passivate the sensor at the same time with the aim at optimizing sensor preparation time. A method describes how the blood sample is delivered to the microfluidic chip. A method is to prevent polymer swelling to increase the performance of the biosensor in aqueous environments, especially based in cavity-coupling. In this case, aluminum oxide thin film coating is used.

A method involves adding a control measurement, in parallel with the biosensing of the target biomarker. A method, based on the methods previously described, involves the detection of multiple biomarkers from the same sample with the aim at identifying an infection with high confidence. A method involves the integration of this sensor with the optical readout system that allows the implementation of portable biosensing system.

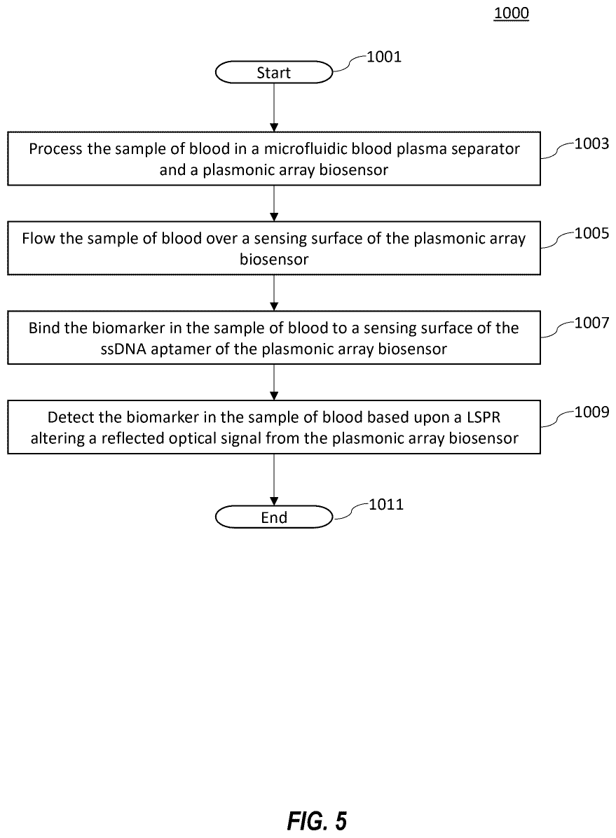

Referring now to FIGS. 1A-1B, 2A, and 5, a method for detecting a biomarker (i.e., a protein caused by an ongoing infection from a virus, a pathogen, a bacterium, or other micro-organism, e.g.) within a sample of blood is now described with reference to a flowchart 1000, which begins at Block 1001. For example, the biomarker may comprise at least one of a DENV1-NS1 protein, a DENV2-NS1 protein, or a Dengue virus protein. In some embodiments, the method may further comprise detecting the biomarker in the sample of blood at a concentration less than 0.2 μg/mL.

The method illustratively includes processing the sample of blood with a biosensor 200. (Block 1003). The biosensor 200 illustratively includes a substrate 201, a microfluidic blood plasma separator 202 on the substrate, and a plasmonic array biosensor 203 on the substrate. The microfluidic blood plasma separator 202 illustratively comprises a first inlet 204a for receiving the sample of blood, a second inlet 204b for receiving a buffer solution, a first outlet 205a for outputting the sample of blood, and a second outlet 205b for outputting the buffer solution.

As perhaps best seen in FIG. 2A, the plasmonic array biosensor 203 illustratively comprises a dielectric layer 210 (e.g., dielectric epoxy, such as SU-8 photoresist), a membrane layer 211 (e.g., Aluminum oxide) over the dielectric layer, and a conductive layer 212 (e.g., gold) over the membrane layer. The plasmonic array biosensor 203 comprises an ssDNA aptamer layer 213 over the conductive layer to define a sensing surface.

The method illustratively comprises flowing the sample of blood over a sensing surface of the plasmonic array biosensor 203. (Block 1005). The sensing surface of the plasmonic array biosensor 203 has an ssDNA aptamer against the biomarker. The method further includes binding the biomarker in the sample of blood to the ssDNA aptamer of the plasmonic array biosensor 203. (Block 1007). The method further comprises reducing nonselective binding from proteins in the sample of blood based upon a passivation layer on the plasmonic array biosensor 203.

The method also includes flowing a buffer solution and the sample of blood through the microfluidic blood plasma separator 202 until the reflected optical signal stabilizes. The method further includes increasing a flow of the sample of blood until plasma separation occurs to provide a plasma sample from the sample of blood, and performing the detection on the plasma sample. The method comprises incubating the plasma sample from the blood sample, and passing the plasma over the sensing surface of the plasmonic array biosensor 203. The method comprises, subsequently to the passing, flushing the microfluidic blood plasma separator 202 with the buffer solution.

The method also includes detecting the biomarker in the sample of blood based upon a LSPR altering a reflected optical signal from the plasmonic array biosensor 203. (Block 1009). In particular, the detection comprises shining an optical signal into the plasmonic array biosensor 203 and detecting the reflected optical signal. The method ends at Block 1011.

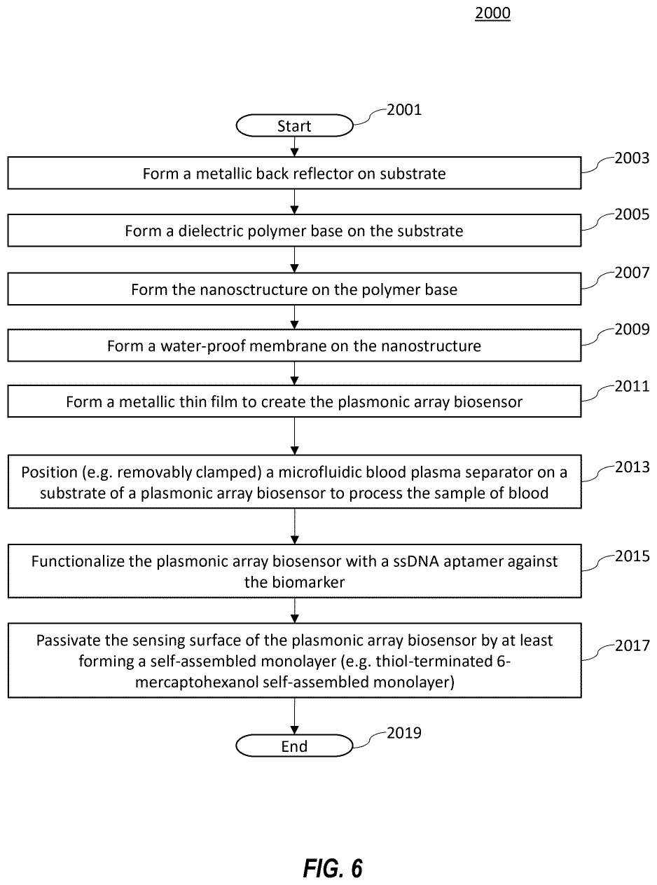

Referring now to FIGS. 1A-1B and 6, a method for making a plasmonic array biosensor for detecting a biomarker within a sample of blood is now described with reference to a flowchart 2000, which begins at Block 2001.

The method illustratively includes forming a metallic back reflector on a substrate 201. (Block 2003). The method comprises forming a dielectric polymer base 210 on the metallic back reflector. (Block 2005). The method comprises forming a nanostructure on the polymer base 210. (Block 2007). The method comprises forming a waterproof membrane 211 on the nanostructure. (Block 2009). For example, the waterproof membrane 211 may comprise aluminum oxide. The method comprises forming a metallic thin film 212 to create the plasmonic array biosensor 203. (Block 2011).

The method illustratively includes positioning a microfluidic blood plasma separator 202 on a substrate 201 of a plasmonic array biosensor 203 to process the sample of blood. (Block 2013). In some embodiments, the microfluidic blood plasma separator 202 may be removably clamped onto the substrate 201, and other embodiments, it may be attached via an adhesive layer.

The method illustratively comprises functionalizing the plasmonic array biosensor 203 with an ssDNA aptamer against the biomarker. (Block 2015). A sensing surface of the plasmonic array biosensor 203 is to bind to the biomarker in the sample of blood. The plasmonic array biosensor 203 is to detect the biomarker in the sample of blood based upon an LSPR signal.

The method further comprises passivating the sensing surface of the plasmonic array biosensor 203 by at least forming a self-assembled monolayer. (Block 2017). For example, the self-assembled monolayer may comprise a thiol-terminated 6-mercaptohexanol self-assembled monolayer.

The plasmonic array biosensor 203 comprises a hole-disc array 206. For example, the hole-disc array 206 may have period of 0.5-0.6 μm, a diameter of 0.1-0.3 μm, and a relief depth of 0.2-0.4 μm. The method ends at Block 2019.

In the following, a discussion of an example embodiment of a method for making the plasmonic array biosensor 203. Glass slides (3″×1″) are cut into square-shaped (1″×1″) slides using a diamond scriber. Each glass slide is thoroughly cleaned with acetone, isopropyl alcohol, and deionized water, and then dried with nitrogen gas. A thin layer of titanium (5 nm) followed by gold (100 nm) is deposited onto the clean glass substrate using electron-beam deposition. An epoxy-based negative photoresist SU-8 2000.5 used to create the dielectric cavity, is spin-coated at 3000 rpm for one minute onto the substrate to achieve the desired thickness (˜700 nm). The substrate is then pre-baked at 95° C. for a minute. A polydimethylsiloxane (PDMS)-based stamp, containing the inverse nanopatterned array structure, is used to thermally emboss the hole-disk array onto the substrate. The substrate is then exposed under UV light (365 nm) for 5 minutes to crosslink and cure the photoresist. A thin 20 nm conformal coating of aluminum oxide (Al₂O₃) is deposited on the embossed substrate via atomic layer deposition. This prevents water diffusion into the polymer that may cause swelling. The substrate is finally coated with a thin film of titanium (3 nm) and gold (30 nm) using e-beam evaporation to create the top plasmonic nanopatterned structure.

In the following, a discussion of an example embodiment of a method for making the microfluidic blood plasma separator 202. The microfluidic blood plasma separator 202 may be fabricated using the standard soft lithography technique. In particular, a dark field mask containing the microfluidic channel pattern is printed onto a transparent sheet. SU-8 2050 is spin-coated on a pristine silicon wafer, previously treated with hexamethyldisilazane (HDMS) adhesion promoter, at 3000 rpm for one minute to produce ˜50 μm thick layer, followed by solvent evaporation at 65° C. for 3 minutes and at 95° C. for 8 minutes. UV-lithography is performed onto the coated wafer, followed by a post-exposure baking at 95° C. for 5 minutes. The wafer is developed in propylene glycol monomethyl ether acetate for 5 minutes followed by isopropyl alcohol and deionized water rinse and dried with nitrogen gas. The wafer is fluorinated with tridecafluoro-1,1,2,2-tetrahydrooctyl triethoxysilane to make the surface hydrophobic. Finally, the microfluidic chip is fabricated using polydimethylsiloxane (PDMS) elastomer mixed with its curing agent in a 10:1 % w/w ratio and poured over the fluorinated silicon master mold. Once degassed, the PDMS-silicon mold is baked at 75° C. in a convection oven for 2 hours, demolded, cut, and punctured with the appropriate inlets and outlets. The microfluidic chip is laminated onto the sensor's surface and kept in a place using acrylic clamps. After each assay, the microfluidic chip was thoroughly cleaned in deionized water, isopropyl alcohol and followed by 10 minutes of sonication in acetone.

In the following, a discussion of an example embodiment of the step for functionalizing the plasmonic array biosensor 203 with an ssDNA aptamer against the biomarker. The sensors are cleaned with ethanol and deionized water, and then dried with nitrogen gas prior to functionalization. Hydrophilic, about 1 mm thick, PDMS film with 3 mm hole is placed on top of each sensor to contain 2 μL of aptamer solution. Hydrophilicity of PDMS surface was introduced by surface modification with polyvinyl alcohol (PVA). In brief, the PDMS is activated in an oxygen plasma chamber for 5 min and incubated in 0.1% w/v aqueous solution of PVA. The petri dish containing the samples is sealed with parafilm and placed on a shaker at 200 rpm for 4 hours. Post-incubation period, each sensor is washed with PBS buffer three times to remove any unbound aptamers. Surface passivation: right after surface functionalization, 2 μL of 30 μM methanolic solution of 6-mercaptahexanol is added to each sensor. The petri dish is re-sealed with parafilm and stored at 4° C. at least 12 hours. The sensors are cleaned with PBS buffer three times to remove unreacted MCH. 2 μL of PBS is added to each sensor, thereafter, to keep them hydrated until used.

### Introduction

Neurotransmitters regulate neural function and well-being in animals, requiring a balanced interplay of neurological hormones for proper bodily function. Among these, dopamine (1) stands out as a critical neuromodulator, playing a pivotal role in regulating cognition (2), emotions such as happiness or pleasure (3, 4), and motor skills (5, 6). Dysregulation of dopamine concentrations in humans are associated with a range of neurodegenerative disorders such as Parkinson's (7) and Alzheimer's disease (8), neurodevelopmental conditions like attention deficit hyperactivity disorder (ADHD) (9) and Tourette syndrome (10), and psychological complications such as bipolar and schizophrenia (11, 12). Additionally, abnormal dopamine levels can serve as a diagnostic indicator for specific types of cancers (13-17). Hence, accurate and reliable detection of dopamine concentrations is critically important for the development of pharmaceutical drug research and medical therapies (18).

Conventional techniques utilize antibody-based enzyme-linked immunosorbent assay (19, 20) (ELISA) or high-performance liquid chromatography (HPLC) for dopamine isolation, coupled with detection methodologies including fluorescence spectrometry (21), colorimetric analysis (22), mass spectrometry (23), or electrochemical reactivity (24-27). These methods, however, suffer from complexities in assay preparation, feasibility, long response time, and selectivity, making them unsuitable for point-of-care applications. Moreover, detecting dopamine directly from unprocessed whole blood with high accuracy and specificity poses significant challenges, primarily due to its low concentration and the presence of interfering molecules (28). Standard detection methods often employ indirect approaches targeting major dopamine metabolites like homovanillic acid (HVA) (29), or utilize catecholamine tests, which measure combined levels of dopamine, epinephrine, and norepinephrine (30, 31), but lack specificity and require complex sample preparation.

In recent decades, electrochemical methods, such as cyclic voltammetry, have emerged as promising tools for the direct, label-free detection of dopamine owing to its simplicity, low cost, and rapid response (32). These methods involve modifying electrodes with various sensing enhancers, offering a viable alternative for detection (33-36). However, since dopamine and several other structurally related catecholamine neurotransmitters have similar redox potentials (37), achieving selectivity remains a major challenge, particularly in complex matrices such as blood or cerebrospinal fluid in the brain. Moreover, an electroactive environment can generate unwanted oxidation products, leading to biofouling of the electrodes, ultimately resulting in poor performance and sensor instability (38). Optical sensors are a strong candidate in the sensitive and label-free detection of such small molecules, with many such studies been shown utilizing photonic, plasmonic or optoelectronic response for quantitative analyses (39-42). These systems, however, though versatile, still suffer from issues such as selectivity, reproducibility, and reliability.

Aptamers, artificial bioreceptors, have emerged as excellent candidates for the specific detection of several neurotransmitters (43, 44), including dopamine. These are usually short, single-stranded RNA or DNA (ssRNA or ssDNA) based oligomer proteins, having a particular nucleotide sequence that can selectively bind to a specific target molecule with high affinity (45, 46). Aptamers can target a wide variety of ligands (47), ranging from simple ions and small molecules, such as neurotransmitters, to large macromolecules like proteins, peptides, viruses, and even whole cells (48). Aptamers are typically selected in-vitro from an RNA or DNA pool via SELEX (systematic evolution of ligands by exponential enrichment) procedure (49, 50). Due to ease of synthesis, good shelf life, and high specificity, they have found applicability as receptors in biosensors for clinical diagnosis and therapy. Recent studies have shown several dopamine-specific aptamers being developed (51), with the first one being an RNA-based aptamer (52). However, due to limited stability and difficulty in synthesis of RNA (53), a DNA homolog (57 base-pair) was subsequently developed to enhance specificity and affinity for dopamine (54). More recently, a shorter DNA aptamer (44 base-pair), obtained through direct selection (55), has been reported to exhibit even higher affinity and selectivity (56). Although, both DNA aptamers have shown potential for dopamine detection across various studies (57-60), recent contradictory results (61) regarding their specificity have complicated efforts toward a comprehensive understanding.

The present disclosure assesses the performance of an all-optical, surface-functionalized plasmonic biosensing platform for the detection of low concentrations of neurotransmitter dopamine directly from diverse biological samples, including protein solutions, artificial cerebrospinal fluid, and unprocessed whole blood. The proposed sensor exhibits highly sensitive narrowband hybrid plasmonic resonances that is tunable across the visible to near-infrared (NIR) spectral range, making it extremely responsive to local alterations as shown in earlier works (48, 62, 63). The sensor surface is functionalized with dopamine-specific aptamers, followed by a passivation procedure to mitigate unwanted charge-induced biofouling and non-specific bindings. Here, the efficacy of two distinct dopamine-specific ssDNA aptamers are compared, named 57-mer and 44-mer based on their respective base-pair lengths, to determine their suitability for detecting dopamine concentrations in general PBS solutions. The results reveal superior binding affinity and sensitivity of the 44-mer at high concentrations compared to the 57-mer counterpart. The present disclosure experimentally demonstrates a broad detection range spanning several orders of magnitude, with a sub-nanomolar detection limit in standard 1× PBS solution, BSA solution and artificial cerebrospinal fluid (aCSF). All these cases demonstrate a strong concentration-dependent signal correlation with minimal interference. Additional tests exhibit good selectivity against dopamine-related catecholamines and metabolites. Finally, integrating the optical biosensing platform with a flow-based microfluidic channel setup allows real-time monitoring of dopamine levels directly from unprocessed whole blood based on a layout reported earlier (63), achieving a detection limit in the range of 1 nM. The versatility of the proposed integrated platform holds promise for simultaneous real-time detection of several neurotransmitters with excellent selectivity. The present disclosure envisions that such aptamer-integrated optical biosensors will serve as a robust platform for label-free, non-invasive, and real-time detection of neurotransmitters with exceptional specificity and sensitivity, with minimal interference, revolutionizing biomedical/clinical diagnostics and monitoring.

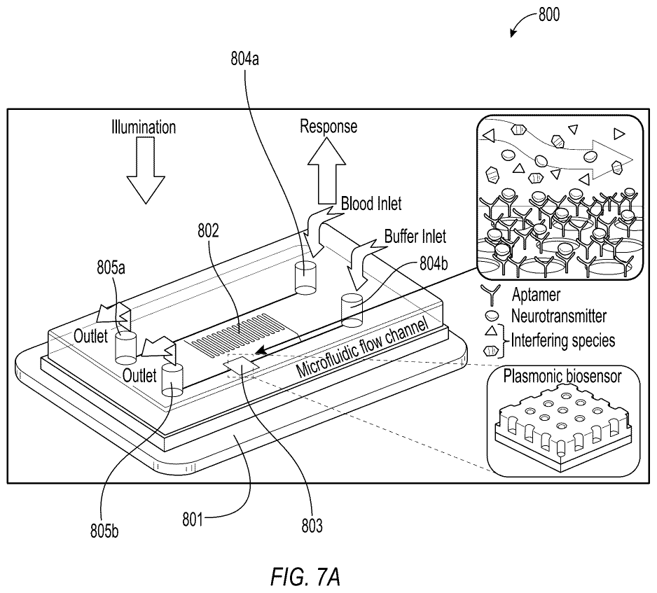

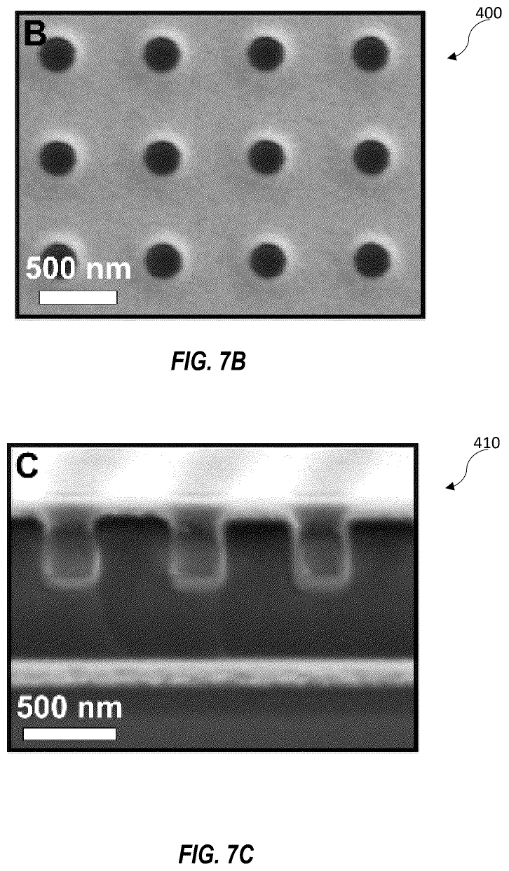

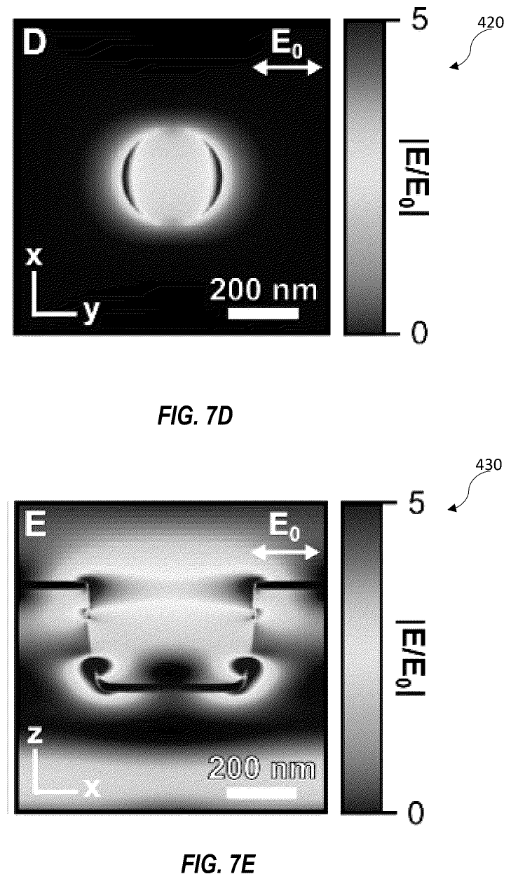

The biosensor 800 includes a surface-functionalized, highly sensitive plasmonic sensor integrated with a polydimethylsiloxane (PDMS)-based microfluidic chip for detection in physiological fluids as shown in FIG. 7A. The plasmonic sensor consists of an array of three-dimensional (3D) hole-disk arrangement of optically-thin metal that is asymmetrically coupled to an underlying resonant cavity with a reflector underneath as shown in previous works (48, 62-65). FIGS. 7B and 7C (images 400, 410) presents scanning electron microscope (SEM) images of the nanostructured surface (top) and vertical cross-section (bottom) of a fabricated plasmonic biosensor respectively, providing detailed insights into its morphology and structure. The nanostructured surface is fabricated using a large-area parallel nanoimprinting method, which enables fabrication of robust and reliable sensors exhibiting multiple hybrid plasmonic resonances in the visible-near infrared (vis-NIR) spectral region (64, 65). This is due to the strong coupling between the top localized surface plasmon (LSP) mode and the photonic cavity mode, resulting in strong, narrowband hybrid resonances that are highly sensitive to local changes in effective refractive index. Previous works have shown the reliable replication of narrowband plasmonic responses, which can be enhanced through automated imprinting methods (48,63).

The cavity-coupled plasmonic biosensor exhibits multi-fold enhancements in the local electric field intensity over the nanostructured surface at the spectral hybrid plasmonic resonances. The finite difference time domain (FDTD) simulated electric near-field magnitude profiles at different perspectives at one such localized surface plasmon resonance (LSPR wavelength =842 nm) are shown in FIG. 7D (diagram 420, top) and 7E (diagram 430, cross-section). The electric field mode in FIG. 7D (x-y plane) appears dipolar due to the polarization of the incident excitation being linear in the x-y plane (64). The cavity is composed of a UV-cured negative epoxy resist SU-8, which offers structural rigidity. To mitigate epoxy swelling during biosensing applications which can induce unpredictable and irreversible changes in spectral information (66), a conformal and uniform coating of 20 nm aluminum oxide (Al₂O₃) layer is deposited using atomic layer deposition (ALD) after nanoimprinting, which prevents undesired fluid diffusion into the epoxy. Additionally, the plasmonic surface is made of optically thin gold (30 nm) coated via electron-beam deposition, which ensures inertness and thus exhibits negligible oxidation and reactivity in almost all environmental conditions (48).

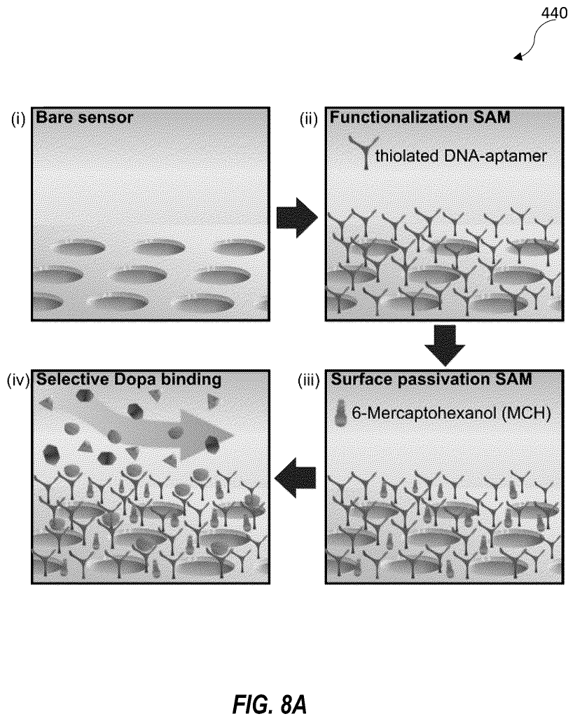

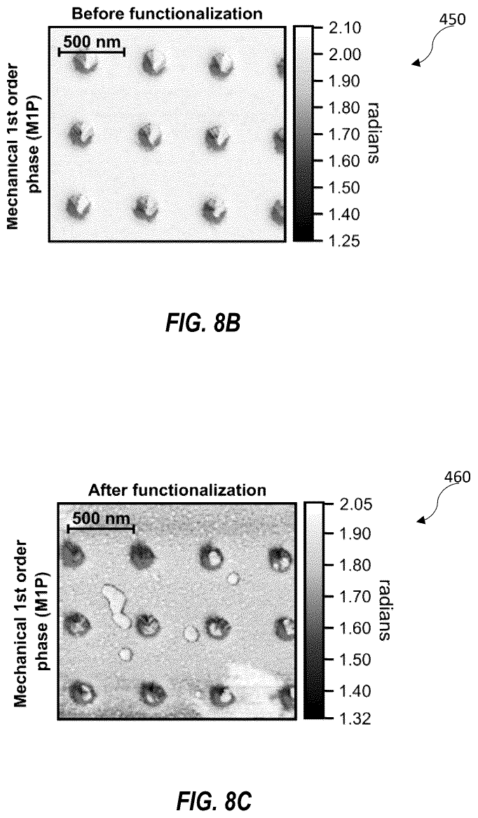

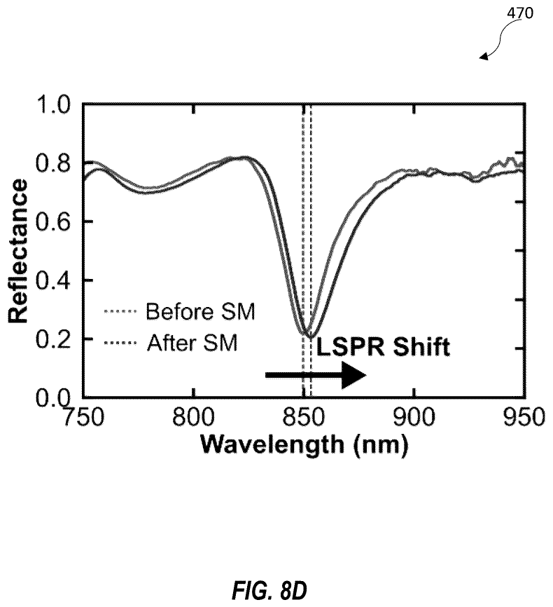

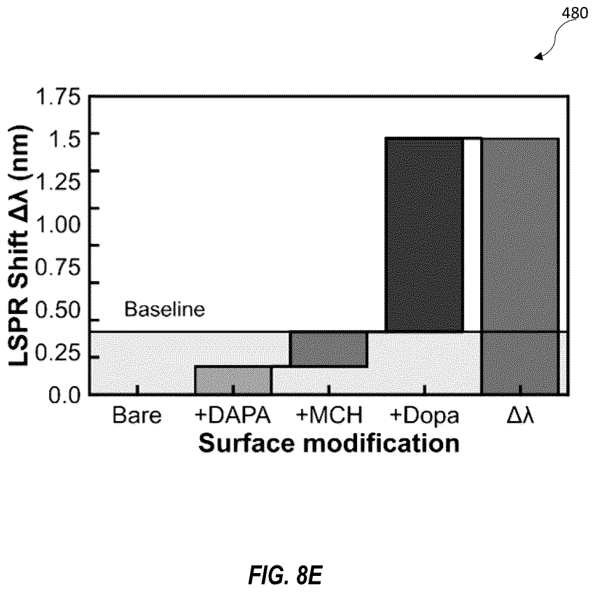

In the first study, it was determined that the functionalizing assay (67) appropriate for low-concentration dopamine detection. For this, two custom-modified thiol-terminated (—S═S—) single-stranded DNA (ssDNA) based aptamer (Integrated DNA Technologies, Inc) of different base-pair lengths, one having 57-base pair and the other having 44-base pairs, are chosen. The two custom ssDNA aptamers, named according to their base-pair lengths as ‘57-mer’ and ‘44-mer’ have similar storage conditions, activation, and surface functionalization protocols. FIG. 8A shows a diagram 440 of the overall strategy for surface modification and detection. The pristine sensors are initially functionalized with the thiol-activated aptamers that covalently bond to the gold, creating a dopamine-specific selective monolayer. Even so, detecting biomarkers in unfiltered or minimally purified bio-samples (blood, plasma matrix, or cerebrospinal fluid) can be challenging due to electrostatic accumulation of proteins on the negatively charged gold surface leading to unwanted biofilm adsorption. This can hinder the actual positive signal acquired because of the desired biomarker-selective binding. To mitigate this issue, a surface passivation technique based on self-assembled monolayer of thiol-terminated 6-mercapto-1-hexanol (6-MCH) is subsequently coated on the surface. This approach is a common strategy for passivating gold-based platforms (67), helping to alleviate the problem of protein biofouling. Following the surface modification, the system's far-field reflectance response undergoes redshifts (FIG. 8C) slightly, which is taken as the baseline for the target analyte detection. After the sensor's functionalization with thiolated ssDNA aptamers and surface passivation with MCH chemistry, the activated system is ready for subsequent measurements and tests pertaining to aptamer selection and dopamine detection via optical spectroscopy and analysis. FIG. 8B presents the biosensor surface topography imaged using atomic force microscopy (AFM). The surface profile here is represented by the first-order mechanical phase amplitude (M1P) of the AFM tip, which carries information regarding the “softness” of the surface. Due to minimal thickness of the surface variations following aptameric functionalization (a few nanometers), they are not discernible in the z-variation profile (68) but a distinct change in surface morphology can be observed in the phase profile (FIGS. 8B-8C, images 450, 460). The detailed interpretation of M1P has been explained in the Methods Section. FIG. 8D includes a diagram 470 of experimental spectra of one of the plasmonic biosensor surface before and after the surface modification events. FIG. 8E includes a diagram 480 of a bridge-plot showing the spectral change for corresponding surface modification events denoted in the x-coordinate.

The primary investigation aims to evaluate the effectiveness of two aptamers in detecting elevated levels of dopamine. For this purpose, several concentrations of dopamine, ranging from 5 mM to 100 mM, in phosphate-buffered saline (1× PBS) was prepared. PBS maintains a consistent pH, ensuring biomolecular stability, and provides controlled ionic strength, mimicking physiological conditions, which is crucial for preserving the proper conformation and function of biomolecules. Additionally, PBS minimizes non-specific interactions and biofouling, enhancing sensor specificity and reliability. Subsequently, these concentrations were incubated separately on two distinct biosensors, one functionalized with a 57-mer aptamer and the other with a 44-mer aptamer. After a 20-minute incubation period, the biosensor surface is rinsed with molecular grade water and air-dried at room temperature for 5 minutes. The thiolated end of the aptamer covalently attaches to the gold surface while the active end selectively binds to the diluted dopamine, inducing a conformational change that generates local variations in the near-field of the biosensing surface. The high sensitivity of the plasmonic sensor's optical near-field to these fluctuations results in a corresponding far-field response, manifesting as a resonance red-shift in the optical spectra. FIGS. 9A-9B shows the corresponding LSPR shifts obtained for the dopamine concentrations in the above-mentioned range. The 57-mer (diagram 490) shows a higher baseline at control compared to the 44-mer (diagram 500), possibly due to being a longer chain oligomer. Furthermore, at higher concentrations, the 57-mer reaches saturation, whereas a linear trend can be seen for the 44-mer. This can be explained by the fact that the LSPR shift being detected is sensitive to near-field changes, and the 57-mer being a long chain does not sufficiently alter the plasmonic near-field after dopamine binding beyond a certain concentration. Therefore, for the subsequent dopamine sensing measurements, the 44-mer aptamer for the sensor functionalization was chosen.

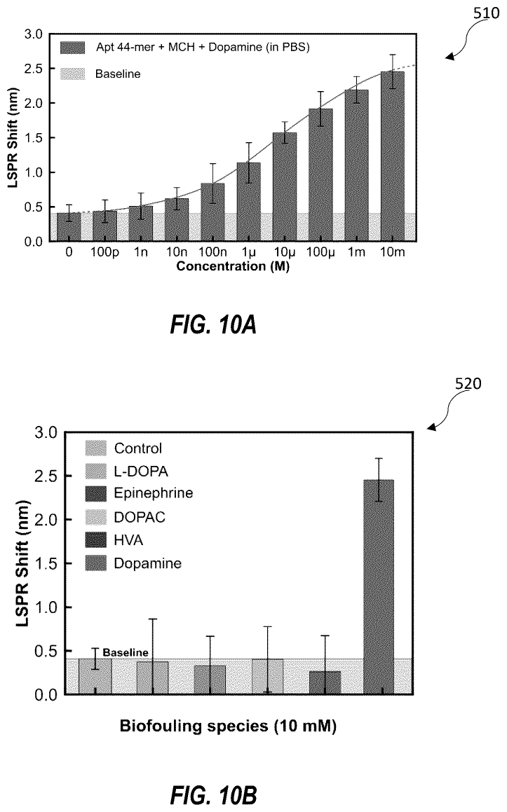

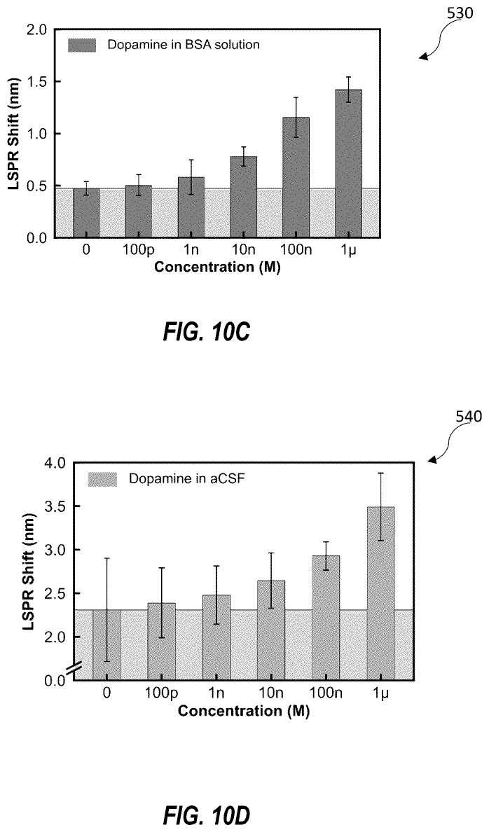

To assess surface coverage for efficient detection of low dopamine concentrations, initially two concentrations of aptamers, 1 μM and 10 μM, shown in diagrams 620, 630 of FIGS. 14A-14B, were tested. For low dopamine concentration (1 nM) in buffered solution, the device with 1 μM aptameric coverage falls within the baseline resolution. The subsequent measurements have therefore been performed with an aptamer concentration of 10 μM. Following this, several different concentrations of dopamine in PBS solution were prepared, and they were incubated onto functionalized sensor surfaces for 20 minutes. The incubated sensors are thoroughly rinsed with molecular-grade water and air-dried, followed by subsequent optical spectra measurements. FIG. 10A shows a diagram 510 of the LSPR shift for various concentrations of dopamine in PBS up to a low concentration of 1 nM. The present disclosure shows a good sigmoidal trend (like a cumulative distribution curve) in the measured LSPR shift with increasing dopamine concentration, shown here up to 10 mM, indicating the wide dynamic concentration range of the measurement setup.

Additionally, dopamine presents several structurally related precursors and metabolites which could potentially interfere with accurate detection and hinder the reliability of the assay. Subsequently, measurements to check for the specificity of the proposed detection platform in the presence of such interfering neurotransmitter species closely related to dopamine were generated. FIG. 10B shows a diagram 520 of the LSPR shift obtained on incubation of the biosensor with several potential interfering neurotransmitter molecules like L-3,4-dihydroxyphenylalanine (L-DOPA), epinephrine, 3,4-dihydroxyphenylacetic acid (DOPAC) and homovanillic acid (HVA), prepared at a concentration of 10 mM in PBS, following a similar assay protocol as the prior experiments. A baseline control measurement is established by incubating unperturbed PBS on the biosensor. The observed shift for the equivalent concentration of dopamine in PBS is also depicted. All measured responses for interfering species are consistently at or below the baseline, in contrast to the response observed for a similar concentration of dopamine, demonstrating exceptional specificity and resistance to biofouling of the functionalized and passivated biosensing platform.

In real-world applications, a biosensor's performance is crucial, particularly in dynamic assays containing diverse interfering molecules such as proteins, lipids, blood cells, and neurotransmitters. To evaluate the biosensor's efficacy in such scenarios, assessments were conducted across two different biological matrices: BSA and artificial cerebrospinal fluid (aCSF). BSA, a standard protein with chemical similarity to human serum albumin (HSA), was utilized for its established protein concentration benchmark. Lyophilized BSA was dissolved in PBS buffer to create a 0.1% w/v solution, to which varying concentrations of dopamine were added and drop-casted onto the post-functionalized biosensor surface. Similarly, aCSF spiked with dopamine was drop-casted onto the passivated biosensors. After a 20-minute incubation at room temperature, the biosensors were gently rinsed, air-dried, and subjected to optical spectrum collection.

FIGS. 10C & 10D include diagrams 530, 540 illustrating the LSPR shift for different dopamine concentrations in spiked-BSA and aCSF solutions, respectively. The LSPR shift observed for incubation with unspiked-BSA was approximately 0.47±0.07 nm, slightly higher than in the previous case. In contrast, the LSPR shift for aCSF without dopamine was notably higher at 2.31±0.59 nm. Nevertheless, a consistent increasing trend in LSPR shift with dopamine concentration was observed, indicative of the biosensor's specificity and resistance to surface binding effects. Furthermore, a consistent monotonic trend in the LSPR shift for various dopamine concentrations underscores the biosensor's superior selectivity, with measured detection limits of about 100 pM for BSA and 90 pM for aCSF.

The main hurdle in point-of-care diagnostics and portable biosensor applications lies in effectively detecting biomarkers within unprocessed or minimally processed complex samples like whole blood, where biofouling as well as harsh environmental conditions can disrupt bio-affinity layers, rendering them ineffective for detection. To address this, the present disclosure utilizes a flow-based system incorporating a functionalized plasmonic biosensor integrated with an on-chip, transparent microfluidic flow channel shown in previous works (48, 63). This biosensor 800, as schematically depicted in FIG. 11A, features dual inlets and outlets for introducing a buffer solution and the target detection matrix (e.g., whole blood). In the experimental protocol, bovine whole blood (˜35% hematocrit) is spiked with dopamine in PBS solution to generate varying dopamine concentrations while maintaining consistent hematocrit levels (refer to Materials and Methods for detailed procedures). Control samples consist of whole blood mixed with an equivalent volume of unspiked PBS.

To initiate the experiment, PBS buffer is first introduced into the functionalized sensor region via one inlet at a low flow rate until the LSPR signal stabilizes. Subsequently, the PBS channel is sealed, and the blood sample is introduced through the second inlet, covering the sensing region. After a 2-minute flow period, the system is allowed to incubate for 5 minutes. Following this incubation, the PBS solution is reintroduced to remove excess blood components, including blood cells, plasma, and proteins. FIG. 11B includes a diagram 560 illustrating the dynamic evolution of the LSPR signal during these flow steps for varying concentrations of dopamine in blood, including the unspiked blood (control). Throughout the blood flow phase, a significant redshift in the tracked LSPR signal is observed, persisting during the incubation period with occasional perturbations due to dynamic changes in the near-field refractive index resulting from events like the introduction of macromolecular blood cells and temporary loss of spectral tracking.

However, flushing with the PBS buffer restores the LSPR tracking signal, albeit with a slight baseline shift attributable to dopamine binding. This change in the baseline LSPR shift after flushing (FIG. 11B) is indicative of surface modification via dopamine capture by the functionalized aptamers. Notably, the control set, devoid of dopamine, exhibits an extremely small baseline shift (˜0.042±0.031 nm), indicating the exceptional stability and resistance of the biosensing surface against external contamination. FIG. 11B presents the experimentally determined differential baseline LSPR shifts corresponding to different concentrations of spiked dopamine in blood, suggesting a limit of detection within the range of 1 nM. Compared to conventional detection techniques requiring extensive sample preparation, the proposed biosensing platform offers direct and highly specific detection of neurotransmitter dopamine from whole blood within a rapid response time of 5 minutes, paving the path forward for real-time monitoring of dopamine directly from unprocessed blood. FIGS. 15A-15B include diagrams 640, 650 of measured raw spectral reflection data for two cases: in PBS, and in CSF, before and after surface modification, respectively. FIG. 16 includes a diagram 660 of a comparison of aptameric concentration response with dopamine concentration in PBS for 1 μM (left) and 10 μM (right) aptameric concentration on the biosensor surface, according to the present disclosure.

### Discussion

In recent years, there has been an increasing demand for blood-based neurotransmitter detection methods that not only provide sensitive and selective responses but also are affordable, reliable, and user-friendly. Conventional techniques like HPLC and ELISA, paired with various detection methods, are limited by complexities in sample preparation, response time, and selectivity. Additionally, traditional blood detection methods often target major dopamine metabolites like HVA or utilize catecholamine tests, but they lack specificity. Overall, the lack of sensitivity, specificity, long response times, high complexity, and cost render these methods inadequate for point-of-care applications.

In this context, aptamers, as artificial bioreceptors, present a promising avenue for specific neurotransmitter detection. This work presents an all-optical, aptamer-based plasmonic biosensor showcasing exceptional sensitivity and selectivity in detecting the neurotransmitter dopamine. The biosensor is surface-functionalized with a thiol-modified aptamer with remarkable specificity toward dopamine, while a self-assembled passivation layer based on MCH minimizes biofouling. Optical sensing performance comparison between two distinct thiol-modified ssDNA aptamers reveals the shorter-chained 44-mer as the optimal candidate. Additionally, sensing experiments conducted in phosphate-buffered saline solutions and biological matrices like bovine serum albumin-spiked solution and artificial cerebrospinal fluid demonstrate excellent picomolar-level concentration-dependent correlation and minimal cross-interference from similar dopamine-related agents. Furthermore, the biosensor's ability to detect dopamine directly from whole blood, with a detection limit of 1 nM and a rapid response time of 5 minutes, exhibits superior selectivity and sensitivity, underscoring its potential for point-of-care diagnostics and portable biosensor applications. Due to the stability of the thiol-gold covalent bond between the modified aptamer and the sensor surface, sensor reusability is difficult requiring an extensive chemical cleaning protocol, and hence is discarded after every detection measurement. Nonetheless, the ease of fabrication allows mass production of such biosensors in batches, making it robust and reliable. Moreover, the microfluidic integrated measurement protocol developed is capable of measuring neurotransmitter directly and rapidly from minimally processed/unprocessed complex matrices via simple optical response. This platform holds promise for addressing unmet needs in real-time monitoring of neurological biomarkers and improving patient care through early and accurate detection of neurotransmitter imbalances.

### Materials and Methods: Biosensor Fabrication

Inverse PDMS stamp fabrication: As an initial step, a pristine silicon wafer is cut and cleaned with acetone, isopropyl alcohol, and deionized water, followed by nitrogen blow-dry. A thin layer of electron-resist (PMMA C4, Kayaku Advanced Materials) is spin-coated on the wafer to generate a thickness of about 350 nm. An electron-beam lithography system (Raith Nanofabrication GmbH) is used to generate the nanostructured hole-disk pattern onto the coated wafer, followed by post-baking at 180° C. and then developed in MIBK/IPA (1:3) developer for 50 seconds to generate a master design pattern. This master pattern is then used to create an inverse poly-dimethyl siloxane (PDMS) based stamp which would be used for subsequent nanoimprint lithography.

Plasmonic sensor fabrication: The fabrication protocol for the biosensors begins with fused silica glass slides as the base substrate. These glass slides are cut into square shapes and sonicated in an acetone bath for 1 hour, following which they are cleaned with isopropyl alcohol and deionized water, and blow-dried with inert nitrogen gas. The pristine glass slides are then coated with a thin layer of 5 nm titanium (Ti) as an adhesion layer, followed by 100 nm of gold (Au) via electron-beam (ebeam) evaporation (AJA International Inc.), to create the reflector base. This is followed by spin-coating of a negative-toned photoresist SU-8 2000.5 (Kayaku Advanced Materials) to generate a dielectric cavity (˜760 nm). The substrate is then pre-baked at 95° C. for 1 minute. The nanostructured pattern is thermally nanoimprinted onto the SU-8 using the PDMS inverse stamp fabricated before. This is followed by an exposure under UV light (365 nm) for 5 minutes, to crosslink and cure the patterned photoresist. A 20 nm conformal layer of aluminum oxide (Al₂O₃) is deposited on the nanopatterned device using atomic layer deposition (ALD, Ultratech Savannah S200). Finally, the substrate is coated with a 3 nm Ti and 30 nm Au via ebeam evaporation to generate the 3D separated hole and disk metallic pattern. This concludes the fabrication process of the plasmonic biosensor, which enables the production of many samples in one batch. See FIGS. 13A-13B, diagrams 600, 610. FIG. 17 includes a diagram 670 of an example setup.

### Biosensor Surface Functionalization and Passivation Protocols

Materials and Reagents: The thiol-modified single-stranded DNA (ssDNA) aptamers were custom-synthesized and received from Integrated DNA Technologies, Inc. The two aptamers, named aptly based on their base-pair lengths as 57-mer and 44-mer have the following base-pair sequences respectively: (1) 5′-/ThioMC6-D/-GTC TCT GTG TGC GCC AGA GAC ACT GGG GCA GAT ATG GGC CAG CAC AGA ATG AGG CCC, (2) 5′-/ThioMC6-D/CGA CGC CAG TTT GAA GGT TCG TTC GCA GGT GTG GAG TGA CGT CG-3′. Both aptamers were centrifuged at 1000 rpm for 5 minutes and each of them were divided into 3μL aliquots of 100 μM concentration, after which they were stored in the freezer at −20° C. until further use. Sodium chloride (NaCl, ≥99%), sodium phosphate dibasic (Na₂HPO₄, ≥99%), potassium phosphate monobasic (KH2PO4, ≥99%), magnesium chloride (MgCl2, >95%), 6-mercapto-1-hexanol (6-MCH, 99%), hydrolyzed polyvinyl alcohol (PVA, ≥99%), tris (2-carboxyethyl) phosphine hydrochloride (TCEP) and dopamine hydrochloride powder ((HO)2C6H3CH2CH2NH2·HCl) were all purchased from Sigma Aldrich. Potassium chloride (KCl, ≥99%) was purchased from Fisher Scientific. Potassium hydroxide (KOH, 10N) was procured from LabChem (TCP Analytical group). Molecular grade water was used for all experiments and was purchased from InterMountain Life Sciences. Bovine serum albumin (lyophilized powder, ≥96%) was purchased from Sigma Aldrich. Artificial Cerebrospinal Fluid (aCSF, sterile) solution was purchased from BioChemazone, for which the pH was verified to be ˜7.36. Bovine Whole blood (in Sodium EDTA) was purchased from Lampire Biological Laboratories.

Aptamers and buffers preparation: The PBS solution was freshly prepared by diluting NaCl, KCL, Na₂HPO₄ and KHPO₄ in the standard ratio in molecular grade water. The pH was adjusted to be about 7.4±0.1. The folding buffer for the aptamers was prepared by adding 2 mM MgCl₂ to PBS. The reducing buffer was prepared by mixing TCEP in cold molecular grade water to achieve an initial stock concentration of 100 mM. The pH is made neutral (˜7.0) by adding 10N KOH. Prior to aptamer reduction, the reducing buffer is diluted with PBS to prepare 10 mM solutions. The protocol for aptamer activation is similar for both 57-mer and 44-mer and is given as follows: A 3 μL frozen aptamer aliquot is thawed at room temperature, after which it is diluted with the folding buffer to prepare a concentration of 10 μM. This mixture is placed in a water bath set at 95° C. for 5 minutes to induce aptamer folding. The mixture is then removed and allowed to cool down at room temperature for 10 minutes. It is diluted further with the reducing buffer in a 1:1 ratio and incubated for 15 minutes to allow thiol-reduction. The final working concentration of the aptamer solution is 10 μM.

Surface functionalization and passivation: Prior to functionalization, the biosensors are cleaned with ethanol and dried with nitrogen gas, followed by 2 minutes of oxygen plasma cleaning in a plasma chamber. Thick PDMS films (˜2 mm) with 3 mm diameter holes were prepared for aptamer confinement over the sensor surface. The PDMS surface was made hydrophilic by activation in an oxygen plasma chamber. The PDMS wells were then placed on top of the sensors and 4μL of aptamer solution was drop-casted in them. The samples are then sealed and allowed to incubate for 4 hours. After incubation, the sensor surface is washed with molecular grade water three times to remove excess unbounded aptamers. After the functionalization procedure, 4μL of 1 mM ethanolic solution of 6-mercapto-1-hexanol (6-MCH) is added to each sensor region for surface passivation. The samples are re-sealed in a petri-dish and stored at 4° C. for 1 hour. After passivation, the unbounded 6-MCH were removed by cleaning the surface three times with molecular grade water. This concludes the functionalization and passivation protocol. The functionalized sensor surface is kept hydrated with a small amount of binding buffer until it is used for the bio-detection experiment.

### Experimental Characterization and Analysis

Sample preparation: Dopamine hydrochloride powder is diluted in PBS solution to make an initial concentration of 100 mM. This is further serially diluted with PBS to prepare subsequent lower order of magnitude concentrations. The precursors and metabolites of dopamine (L-DOPA, epinephrine, 3,4-dihydroxyphenylacetic acid (DOPAC) and homovanillic acid) were all prepared accordingly by dilution in PBS. For dilution in BSA, an initial concentration of 100 μM of BSA in molecular grade water is prepared. This is further serial diluted with dopamine in PBS solution to achieve the final appropriate concentration. Similarly, dopamine is diluted in aCSF (as purchased) to create an initial concentration of 100 mM, which is further serially diluted in aCSF to generate subsequent lower concentrations. For dopamine solutions in whole blood, 30 μL of 10 μM dopamine in PBS was mixed with 270 μL of whole blood, to generate 1 μM concentration of dopamine in blood. The same protocol was followed to prepare all subsequent concentrations of dopamine, including the control solution, for which unspiked PBS was mixed with whole blood.

Optical characterization: A single illumination wavelength intensity-based detection schemes do not provide nano to pico-molar level detection resolution of small molecules like dopamine. A spectrometer (or plate-reader in medical terminology) is a common medical device nowadays present in most diagnostic centers/clinics/hospitals making spectroscopic detection a viable, accurate and technologically relevant solution. For the optical measurements, the present disclosure used a custom-build setup comprising of a grating spectrometer (HR2000+, Ocean Optics) with a 0.035 nm spectral resolution, in reflection mode. The LSPR in the collected reflectance spectra is fitted with a quadratic function around the tracked resonance. The sensor's LSPR shift response is calculated by subtracting the sensor's initial LSPR response from the final LSPR response achieved after biofluid drop-casting, incubation, and flushing. The error bars shown on the bar charts (FIGS. 9A-9B & 11A-11C, diagrams 490, 500, 560, 570) are calculated by the standard deviation of multiple spectral LSPR shifts measured. The measurement for each dopamine concentration in the fluid matrix has been performed at least 5 times. For the dopamine spiked in aCSF based LSPR shifts, measurements were performed at least 8 times for each dopamine concentration prior to averaging. FIG. 12A includes a diagram 580 of a comparison of experimental and simulated reflectance response of the plasmonic biosensor. FIG. 12B includes an image 590 of a plasmonic biosensor.

Microfluidic Integration: For microfluidic measurements, a PDMS-based transparent microfluidic chip, fabricated using a standard soft-lithography technique, is laminated onto the functionalized biosensor's surface. Prior to lamination, the chip's surface is treated in oxygen plasma for 4 minutes to promote adhesion and induce hydrophilicity. The chip is then affixed in place using an acrylic clamp. A customized optical setup integrated with a flow-based system is used. The optical setup consists of the same grating spectrometer in reflection configuration, being utilized with a LabVIEW (National Instrument)-based spectra logging and resonance tracking software, which collects reflectance spectra dynamically every second. The software also controls the integrated flow system (ElveFlow Microfluidics) that redirects the desired fluids through the microfluidic channels over the measurement region. After each measurement, the microfluidic chip was thoroughly cleaned in deionized water and ethanol, followed by 10 minutes of sonication in acetone, and stored in scotch tapes for further measurements.

### Atomic Force Microscopy (AFM) Measurements

The surface morphology experiments were performed by an atomic force microscopy (AFM) setup (NeaSpec GmbH). In addition to the surface topography mapping, first-order mechanical amplitude (M1A) and phase (M1P) maps were simultaneously acquired by the system. The M1P maps are useful in identifying separate sample species showing inhomogeneity in adhesion and stiffness (68). Sub-nanometer thin layer of aptamers on the gold surface can be readily isolated from the M1P maps in contrast to the topography maps, where the height variations are close to the surface roughness.

### Simulations

The present disclosure includes numerical simulations to determine the reflectance spectra and spatial electromagnetic profile at resonance using a finite-difference time domain-based software (Ansys Lumerical FDTD). The simulation geometry consists of multiple stacked layers: an initial 100 nm gold backside reflector, a dielectric cavity having variable thickness denoted as L. The top surface of the cavity consists of a hole, generated as a custom surface to replicate the actual depressed hole as seen in the SEM images. The relief depth and diameter of the hole were both kept fixed at 300 nm. The hole was coated conformally with a 20 nm layer of aluminum oxide (Al₂O₃). Finally, a 30 nm gold-based separated disk and hole is generated on the top. The system period is kept fixed at 580 nm. Anti-symmetric boundary conditions were applied along the x-boundaries and symmetric boundary conditions along the y-boundaries. The refractive index of the dielectric cavity was set at 1.59 for the whole range, equivalent to SU-8's index. The dispersion data for gold and Al₂O₃ were taken from Palik's handbook (69). Two profile monitors were used to calculate the electric field intensity profile at resonance, one vertically through the middle of the unit cell and one horizontally 2 nm above the gold surface of the unit cell.

Referring now additionally to FIGS. 7A & 11A, another embodiment of the biosensor 800 is now described. In this embodiment of the biosensor 800, those elements already discussed above with respect to FIG. 1A are incremented by 600 and most require no further discussion herein. This embodiment differs from the previous embodiment in that this biosensor 800 is used to sense chemical messenger biomarkers in the sample of blood. The biosensor 800 illustratively includes a substrate 801, first and second inlets 804a-804b on the substrate and respectively receiving the sample of blood and buffer solution, a first outlet 805a for outputting the sample of blood, a second outlet 805b for outputting the buffer solution, a microfluidic blood plasma separator 802 on the substrate and receiving the sample of blood and coupled between the first and second inlets, a plasmonic array biosensor 803 on the substrate coupled between the first second inlet and second outlet.

In one aspect, a method is for detecting a chemical messenger within a sample of blood. The method comprises processing the sample of blood with a microfluidic blood plasma separator 802 and a plasmonic array biosensor 803, and flowing the sample of blood over a sensing surface of the plasmonic array biosensor. The sensing surface of the plasmonic array biosensor 803 is functionalized with a ssDNA aptamer against the chemical messenger. The method also includes binding the chemical messenger in the sample of blood to the ssDNA aptamer of the plasmonic array biosensor 803, and detecting the chemical messenger in the sample of blood based upon a LSPR shift altering a reflected optical signal from the plasmonic array biosensor.

In particular, the detecting comprises shining an optical signal into the plasmonic array biosensor 803 and detecting the LSPR shift of the reflected optical signal. The chemical messenger may comprise at least one of dopamine, serotonin, and epinephrine, for example.

Also, the method may also include flowing a buffer solution and the sample of blood through the microfluidic blood plasma separator 802 until the reflected optical signal stabilizes. The processing may comprise receiving the sample of blood and the buffer solution through separate inlets 804a-804b. The method may also include increasing a flow of the sample of blood until plasma separation occurs to provide a plasma sample from the sample of blood, and performing the detecting on the plasma sample. The method may further include incubating the plasma sample from the blood sample, passing the plasma over the sensing surface of the plasmonic array biosensor 803, and subsequently to the passing, flushing the microfluidic blood plasma separator 802 with the buffer solution.

In some embodiments, the method may also include reducing nonselective binding from proteins in the sample of blood based upon a passivation layer on the plasmonic array biosensor 803. The detecting of the chemical messenger in the sample of blood may be performed detecting of the chemical messenger in the sample of blood at a concentration less than 0.2 μg/mL, and in less than 6 minutes.

Another aspect is directed to a method for making a biosensor 800 for detecting a chemical messenger within a sample of blood. The method comprises positioning a microfluidic blood plasma separator 802 on a substrate 801 of a plasmonic array biosensor 803 to process the sample of blood, and functionalizing the plasmonic array biosensor with a ssDNA aptamer against the chemical messenger. A sensing surface of the plasmonic array biosensor 803 is to bind to the chemical messenger in the sample of blood. The plasmonic array biosensor 803 is to detect the chemical messenger in the sample of blood based upon a shift in a LSPR signal, resulting in the LSPR shift in incident probing optical signal reflection.

The method may also include passivating the sensing surface of the plasmonic array biosensor 803 by at least forming a self-assembled monolayer. For example, the self-assembled monolayer may comprise a thiol-terminated 6-mercaptohexanol self-assembled monolayer. In some embodiments, the microfluidic blood plasma separator 802 may be removably clamped onto the substrate. The plasmonic array biosensor 803 may comprise a hole-disc array. For instance, the hole-disc array may have period of 0.5-0.6 μm, a diameter of 0.1-0.3 μm, and a relief depth of 0.2-0.4 μm. Also, the method may include forming a metallic back reflector on the plasmonic array biosensor 903, forming a dielectric polymer base for the plasmonic array biosensor, and forming a waterproof membrane on the dielectric polymer base.

Helpfully, the present disclosure may provide a novel plasmonic sensor platform capable of detecting neurotransmitters, specifically dopamine, directly from whole blood without requiring plasma separation. The disclosed method may use a single-stranded DNA aptamer designed to bind dopamine with high affinity, with minimal interference from other molecules. The sensor platform may be versatile and can target other neurotransmitters, such as serotonin or epinephrine, by changing the aptamer.

The present disclosure builds on prior embodiments used for detecting viral genomes, which required plasma separation for noise reduction. The present disclosure adapts the same sensing mechanism to target neurotransmitters, significantly broadening its application to mental health monitoring.

It should be appreciated that features from each of the disclosed embodiments of the biosensors 200, 800 may be combined with each other. Other features, which may be combined with the present embodiments, for a biosensor may be found in “Nanoplasmonic aptasensor for sensitive, selective, and real-time detection of dopamine from unprocessed whole blood”, Biswas et al. (authored by the inventors of the present application), Sci. Adv. 10, eadp7460 (2024) 4 Sep. 2024, the contents of which are hereby incorporated by reference in their entirety.

Many modifications and other embodiments of the present disclosure will come to the mind of one skilled in the art having the benefit of the teachings presented in the foregoing descriptions and the associated drawings. Therefore, it is understood that the present disclosure is not to be limited to the specific embodiments disclosed, and that modifications and embodiments are intended to be included within the scope of the appended claims.

### REFERENCES

### The Contents of Which are Hereby Incorporated by Reference in Their Entirety

1. K. M. Costa, G. Schoenbaum, Dopamine. Current Biology 32, R817-R824 (2022).
2. A. Nieoullon, Dopamine and the regulation of cognition and attention. Prog Neurobiol 67, 53-83 (2002).
3. E. S. Bromberg-Martin, M. Matsumoto, O. Hikosaka, Dopamine in motivational control: rewarding, aversive, and alerting. Neuron 68, 815 (2010).
4. R. A. Wise, C. J. Jordan, Dopamine, behavior, and addiction. Journal of Biomedical Science 2021 28:1 28, 1-9 (2021).
5. A. N. Wood, New roles for dopamine in motor skill acquisition: lessons from primates, rodents, and songbirds. J Neurophysiol 125, 2361-2374 (2021).
6. A. Bova, M. Gaidica, A. Hurst, Y. Iwai, J. Hunter, D. K. Leventha, Precisely-timed dopamine signals establish distinct kinematic representations of skilled movements. Elife 9, 1-141 (2020).
7. J. Jankovic, Parkinson's disease: clinical features and diagnosis. J Neurol Neurosurg Psychiatry 79, 368-376 (2008).
8. X. Pan, A. C. Kaminga, S. W. Wen, X. Wu, K. Acheampong, A. Liu, Dopamine and dopamine receptors in Alzheimer's disease: A systematic review and network meta-analysis. Front Aging Neurosci 10, 175 (2019).
9. K. Blum, A. L. C. Chen, E. R. Braverman, D. E. Comings, T. J. H. Chen, V. Arcuri, S. H. Blum, B. W. Downs, R. L. Waite, A. Notaro, J. Lubar, L. Williams, T. J. Prihoda, T. Palomo, M. Oscar-Berman, Attention-deficit-hyperactivity disorder and reward deficiency syndrome. Neuropsychiatr Dis Treat 4, 893 (2008).
10. H. S. Singer, I. J. Butler, L. E. Tune, W. E. Seifert, J. T. Coyle, Dopaminergic dysfunction in tourette syndrome. Ann Neurol 12, 361-366 (1982).
11. R. Brisch, A. Saniotis, R. Wolf, H. Bielau, H. G. Bernstein, J. Steiner, B. Bogerts, K. Braun, J. Kumaratilake, M. Henneberg, T. Gos, The Role of Dopamine in Schizophrenia from a Neurobiological and Evolutionary Perspective: Old Fashioned, but Still in Vogue. Front Psychiatry 5(2014 ).
12. K. L. Davis, R. S. Kahn, G. Ko, M. Davidson, Dopamine in schizophrenia: a review and reconceptualization. Am J Psychiatry 148, 1474-1486 (1991).
13. C. E. Grant, A. L. Flis, B. M. Ryan, Understanding the role of dopamine in cancer: past, present and future. Carcinogenesis 43, 517-527 (2022).
14. Y. Yan, J. Pan, Y. Chen, W. Xing, Q. Li, D. Wang, X. Zhou, J. Xie, C. Miao, Y. Yuan, W. Zeng, D. Chen, Increased dopamine and its receptor dopamine receptor D1 promote tumor growth in human hepatocellular carcinoma. Cancer Commun 40, 694-710 (2020).
15. S. Miyamoto, Y. Yoshida, Y. Ozeki, M. Okamoto, K. Gotoh, T. Masaki, H. Nishida, T. Shibuya, T. Shin, T. Daa, H. Mimata, N. Kimura, H. Shibata, Dopamine-Secreting Pheochromocytoma and Paraganglioma. J Endocr Soc 5, 1-9 (2021).
16. A. N. A. V. A. N. D. E. R. HORST-SCHRIVERS, T. E. OSINGA, I. D. O. P. KEMA, B. F. A. M. V. A. N. D. E. R. LAAN, R. P. F. DULLAART, Dopamine Excess in Patients with Head and Neck Paragangliomas. Anticancer Res 30, 5153 (2010).
17. G. Eisenhofer, D. S. Goldstein, P. Sullivan, G. Csako, F. M. Brouwers, E. W. Lai, K. T. Adams, K. Pacak, Biochemical and clinical manifestations of dopamine-producing paragangliomas:
utility of plasma methoxytyramine. J Clin Endocrinol Metab 90, 2068-2075 (2005).
18. D. S. Goldstein, G. Eisenhofer, I. J. Kopin, Sources and significance of plasma levels of catechols and their metabolites in humans. J Pharmacol Exp Ther 305, 800-811 (2003).
19. H. Park, I. R. Paeng, Development of direct competitive enzyme-linked aptamer assay for determination of dopamine in serum. Anal Chim Acta 685, 65-73 (2011).
20. J. Kim, M. Jeon, K. J. Paeng, I. R. Paeng, Competitive enzyme-linked immunosorbent assay for the determination of catecholamine, dopamine in serum. Anal Chim Acta 619, 87-93 (2008).
21. X. Liu, M. Tian, W. Gao, J. Zhao, A Simple, Rapid, Fluorometric Assay for Dopamine by in Situ Reaction of Boronic Acids and cis-Diol. J Anal Methods Chem 2019 (2019 ).
22. L. Liang, Z. Zhao, F. Ye, S. Zhao, Rapid and sensitive colorimetric detection of dopamine based on the enhanced-oxidase mimicking activity of cerium (IV). New Journal of Chemistry 45, 6780-6786 (2021).
23. J. Y. Park, S. W. Myung, I. S. Kim, D. K. Choi, S. J. Kwon, S. H. Yoon, Simultaneous measurement of serotonin, dopamine and their metabolites in mouse brain extracts by high-performance liquid chromatography with mass spectrometry following derivatization with ethyl chloroformate. Biol Pharm Bull 36, 252-258 (2013).
24. G. Cannazza, A. Di Stefano, B. Mosciatti, D. Braghiroli, M. Baraldi, F. Pinnen, P. Sozio, C. Benatti, C. Parenti, Detection of levodopa, dopamine and its metabolites in rat striatum dialysates following peripheral administration of L-DOPA prodrugs by mean of HPLC-EC. J Pharm Biomed Anal 36, 1079-1084 (2005).
25. P. S. Rao, N. Rujikarn, J. M. Luber, D. H. Tyras, A specific sensitive HPLC method for determination of plasma dopamine. Chromatographia 1989 28:5 28, 307-310 (1989).
26. F.-C. Jing, H. Chen, C.-L. Li, Rapid determination of dopamine and its metabolites during in vivo cerebral microdialysis by routine high performance liquid chromatography with electrochemical detection. Biomed Environ Sci 20, 317-20 (2007).
27. S. Sasa, C. L. R. Blank, Determination of Serotonin and Dopamine in Mouse Brain Tissue by High Performance Liquid Chromatography with Electrochemical Detection. Anal Chem 49, 354-359 (1977).
28. A. J. Stewart, J. Hendry, L. Dennany, Whole Blood Electrochemiluminescent Detection of Dopamine. Anal Chem 87, 11847-11853 (2015).
29. G. W. Lambert, G. Eisenhofer, H. S. Cox, M. Horne, V. Kalff, M. Kelly, G. L. Jennings, M. D. Esler, Direct determination of homovanillic acid release from the human brain, an indicator of central dopaminergic activity. Life Sci 49, 1061-1072 (1991).
30. R. N. Goyal, S. Bishnoi, Simultaneous determination of epinephrine and norepinephrine in human blood plasma and urine samples using nanotubes modified edge plane pyrolytic graphite electrode. Talanta 84, 78-83 (2011).
31. Y. Wang, D. S. Fice, P. K. F. Yeung, A simple high-performance liquid chromatography assay for simultaneous determination of plasma norepinephrine, epinephrine, dopamine and 3,4-dihydroxyphenyl acetic acid. J Pharm Biomed Anal 21, 519-525 (1999).
32. Q. Ning, S. Feng, Y. Cheng, T. Li, D. Cui, K. Wang, Point-of-care biochemical assays using electrochemical technologies: approaches, applications, and opportunities. Mikrochim Acta 189, 3 (2022).
33. S. Lakard, I. A. Pavel, B. Lakard, Electrochemical Biosensing of Dopamine Neurotransmitter: A Review. Biosensors (Basel) 11 (2021 ).
34. D. S. Kim, E. S. Kang, S. Baek, S. S. Choo, Y. H. Chung, D. Lee, J. Min, T. H. Kim, Electrochemical detection of dopamine using periodic cylindrical gold nanoelectrode arrays. Scientific Reports 2018 8:1 8, 1-10 (2018).
35. Z. Hsine, R. Mlika, N. Jaffrezic-Renault, H. Korri-Youssoufi, Review—Recent Progress in Graphene Based Modified Electrodes for Electrochemical Detection of Dopamine. Chemosensors 2022, Vol. 10, Page 249 10, 249 (2022).
36. M. M. Rahman, J. J. Lee, Electrochemical Dopamine Sensors Based on Graphene. Journal of Electrochemical Science and Technology 10, 185-195 (2019).
37. A. Suzuki, T. A. Ivandini, K. Yoshimi, A. Fujishima, G. Oyama, T. Nakazato, N. Hattori, S. Kitazawa, Y. Einaga, Fabrication, characterization, and application of boron-doped diamond microelectrodes for in vivo dopamine detection. Anal Chem 79, 8608-8615 (2007).
38. H. S. Wang, T. H. Li, W. L. Jia, H. Y. Xu, Highly selective and sensitive determination of dopamine using a Nafion/carbon nanotubes coated poly(3-methylthiophene) modified electrode. Biosens Bioelectron 22, 664-669 (2006).
39. V. T. N. Linh, M. Y. Lee, J. Mun, Y. Kim, H. Kim, I. W. Han, S. G. Park, S. Choi, D. H. Kim, J. Rho, H. S. Jung, 3D plasmonic coral nanoarchitecture paper for label-free human urine sensing and deep learning-assisted cancer screening. Biosens Bioelectron 224, 115076 (2023).
40. V. T. N. Linh, H. Kim, M. Y. Lee, J. Mun, Y. Kim, B. H. Jeong, S. G. Park, D. H. Kim, J. Rho, H. S. Jung, 3D plasmonic hexaplex paper sensor for label-free human saliva sensing and machine learning-assisted early-stage lung cancer screening. Biosens Bioelectron 244, 115779 (2024).
41. W. G. Kim, J. M. Lee, Y. Yang, H. Kim, V. Devaraj, M. Kim, H. Jeong, E. J. Choi, J. Yang, Y. Jang, T. Badloe, D. Lee, J. Rho, J. T. Kim, J. W. Oh, Three-Dimensional Plasmonic Nanocluster-Driven Light-Matter Interaction for Photoluminescence Enhancement and Picomolar-Level Biosensing. Nano Lett 22, 4702-4711 (2022).
42. I. Kim, H. Kim, S. Han, J. Kim, Y. Kim, S. Eom, A. Barulin, I. Choi, J. Rho, L. P. Lee, Metasurfaces-Driven Hyperspectral Imaging via Multiplexed Plasmonic Resonance Energy Transfer. Advanced Materials 35, 2300229 (2023).
43. J. H. Park, Y. S. Eom, T. H. Kim, Recent Advances in Aptamer-Based Sensors for Sensitive Detection of Neurotransmitters. Biosensors (Basel) 13 (2023 ).
44. K. Sinha, C. Das Mukhopadhyay, Quantitative detection of neurotransmitter using aptamer: From diagnosis to therapeutics. J Biosci 45, 1-12 (2020).
45. N. Saraf, E. R. Woods, M. Peppler, S. Seal, Highly selective aptamer based organic electrochemical biosensor with pico-level detection. Biosens Bioelectron 117, 40-46 (2018).
46. J. Zhou, M. R. Battig, Y. Wang, Aptamer-based molecular recognition for biosensor development. Analytical and Bioanalytical Chemistry 2010 398:6 398, 2471-2480 (2010).
47. Y. Zhang, B. S. Lai, M. Juhas, Recent Advances in Aptamer Discovery and Applications. Molecules 24 (2019 ).
48. A. Vázquez-Guardado, F. Mehta, B. Jimenez, A. Biswas, K. Ray, A. Baksh, S. Lee, N. Saraf, S. Seal, D. Chanda, DNA-Modified Plasmonic Sensor for the Direct Detection of Virus Biomarkers from the Blood. Nano Lett 21, 7505-7511 (2021).
49. C. Tuerk, L. Gold, Systematic Evolution of Ligands by Exponential Enrichment: RNA Ligands to Bacteriophage T4 DNA Polymerase. Science (1979) 249, 505-510 (1990).
50. A. D. Ellington, J. W. Szostak, In vitro selection of RNA molecules that bind specific ligands. Nature 1990 346:6287 346, 818-822 (1990).
51. X. Liu, J. Liu, Biosensors and sensors for dopamine detection. View 2, 20200102 (2021).
52. C. Mannironi, A. Di Nardo, P. Fruscoloni, G. P. Tocchini-Valentini, In vitro selection of dopamine RNA ligands. Biochemistry 36, 9726-9734 (1997).
53. E. Farjami, R. Campos, J. S. Nielsen, K. V. Gothelf, J. Kjems, E. E. Ferapontova, RNA aptamer-based electrochemical biosensor for selective and label-free analysis of dopamine. Anal Chem 85, 121-128 (2013).
54. R. Walsh, M. C. DeRosa, Retention of function in the DNA homolog of the RNA dopamine aptamer. Biochem Biophys Res Commun 388, 732-735 (2009).
55. N. Nakatsuka, K.-A. Yang, J. M. Abendroth, K. M. Cheung, X. Xu, H. Yang, C. Zhao, B. Zhu, Y. S. Rim, Y. Yang, P. S. Weiss, M. N. Stojanović, A. M. Andrews, Aptamer-field-effect transistors overcome Debye length limitations for small-molecule sensing. Science (1979) 362, 319-324 (2018).
56. Y. Hou, J. Hou, X. Liu, Comparison of Two DNA Aptamers for Dopamine Using Homogeneous Binding Assays. ChemBioChem 22, 1948-1954 (2021 ).
57. J. Zhou, W. Wang, P. Yu, E. Xiong, X. Zhang, J. Chen, A simple label-free electrochemical aptasensor for dopamine detection. RSC Adv 4, 52250-52255 (2014).
58. M. Jarczewska, S. R. Sheelam, R. Ziólkowski, L. Górski, A Label-Free Electrochemical DNA Aptasensor for the Detection of Dopamine. J Electrochem Soc 163, B26-B31 (2016).
59. M. Jarczewska, L. Górski, E. Malinowska, Electrochemical aptamer-based biosensors as potential tools for clinical diagnostics. Analytical Methods 8, 3861-3877 (2016).
60. H. Abu-Ali, O. Cansu, F. Davis, N. Walch, A. Nabok, Electrochemical Aptasensor for Detection of Dopamine. Chemosensors 2020, Vol. 8, Page 28 8, 28 (2020).
61. I. Álvarez-Martos, E. E. Ferapontova, A DNA sequence obtained by replacement of the dopamine RNA aptamer bases is not an aptamer. Biochem Biophys Res Commun 489, 381-385 (2017).
62. W. Wilson, D. Chanda, J. M. Perez, J. Ortega, A. Smith, A. Vázquez-Guardado, Hybrid cavity-coupled plasmonic biosensors for low concentration, label-free and selective biomolecular detection. Optics Express, Vol. 24, Issue 22, pp. 25785-25796 24, 25785-25796 (2016).
63. A. Vázquez-Guardado, S. Barkam, M. Peppler, A. Biswas, W. Dennis, S. Das, S. Seal, D. Chanda, Enzyme-Free Plasmonic Biosensor for Direct Detection of Neurotransmitter Dopamine from Whole Blood. Nano Lett 19, 449-454 (2019).
64. A. Vázquez-Guardado, A. Safaei, S. Modak, D. Franklin, D. Chanda, Hybrid coupling mechanism in a system supporting high order diffraction, plasmonic, and cavity resonances. Phys Rev Lett 113, 263902 (2014).
65. D. Chanda, K. Shigeta, T. Truong, E. Lui, A. Mihi, M. Schulmerich, P. V. Braun, R. Bhargava, J. A. Rogers, Coupling of plasmonic and optical cavity modes in quasi-three-dimensional plasmonic crystals. Nature Communications 2011 2:1 2, 1-7 (2011).
66. K. Wouters, R. Puers, Diffusing and swelling in SU-8: insight in material properties and processing. Journal of Micromechanics and Microengineering 20, 095013 (2010).
67. F. V. Oberhaus, D. Frense, D. Beckmann, Immobilization Techniques for Aptamers on Gold Electrodes for the Electrochemical Detection of Proteins: A Review. Biosensors 2020, Vol. 10, Page 45 10, 45 (2020).
68. E. P. S. Tan, C. T. Lim, Effects of annealing on the structural and mechanical properties of electrospun polymeric nanofibres. Nanotechnology 17, 2649 (2006).
69. E. Palik, Handbook of Optical Constants of Solids (Academic press, Volume 3., 1998; https: //books. google. com/books?hl=en&lr=&id=nxoqxyoHfbIC&oi=fnd&pg=PA3&ots=EHXXOBt4gg&sig=w96a6Nzg0nRAnv-Q7bZjol8lv5o).

1. A method for detecting a chemical messenger within a sample of blood, the method comprising:
processing the sample of blood with a microfluidic blood plasma separator and a plasmonic array biosensor;
flowing the sample of blood over a sensing surface of the plasmonic array biosensor, the sensing surface of the plasmonic array biosensor functionalized with a single stranded DNA (ssDNA) aptamer against the chemical messenger;
binding the chemical messenger in the sample of blood to the ssDNA aptamer of the plasmonic array biosensor; and
detecting the chemical messenger in the sample of blood based upon a localized surface plasmon resonance (LSPR) shift altering a reflected optical signal from the plasmonic array biosensor.

2. The method of claim 1 wherein the detecting comprises shining an optical signal into the plasmonic array biosensor and detecting the LSPR shift of the reflected optical signal.

3. The method of claim 1 wherein the chemical messenger comprises at least one of dopamine, serotonin, and epinephrine.

4. The method of claim 1 further comprising flowing a buffer solution and the sample of blood through the microfluidic blood plasma separator until the reflected optical signal stabilizes.

5. The method of claim 4 wherein the processing comprises receiving the sample of blood and the buffer solution through separate inlets.

6. The method of claim 4 further comprising increasing a flow of the sample of blood until plasma separation occurs to provide a plasma sample from the sample of blood, and performing the detecting on the plasma sample.

7. The method of claim 6 further comprising incubating the plasma sample from the blood sample, and passing the plasma over the sensing surface of the plasmonic array biosensor.

8. The method of claim 7 further comprising, subsequently to the passing, flushing the microfluidic blood plasma separator with the buffer solution.

9. The method of claim 1 further comprising reducing nonselective binding from proteins in the sample of blood based upon a passivation layer on the plasmonic array biosensor.

10. The method of claim 1 wherein the detecting of the chemical messenger in the sample of blood is performed at a concentration less than 0.2 μg/mL.

11. The method of claim 1 wherein the detecting is performed in less than 6 minutes.

12. A method for making a biosensor for detecting a chemical messenger within a sample of blood, the method comprising:
positioning a microfluidic blood plasma separator on a substrate of a plasmonic array biosensor to process the sample of blood; and
functionalizing the plasmonic array biosensor with a single stranded DNA (ssDNA) aptamer against the chemical messenger, a sensing surface of the plasmonic array biosensor to bind to the chemical messenger in the sample of blood, the plasmonic array biosensor to detect the chemical messenger in the sample of blood based upon a shift in a localized surface plasmon resonance (LSPR) signal, resulting in the LSPR shift in incident probing optical signal reflection.

13. The method of claim 12 further comprising passivating the sensing surface of the plasmonic array biosensor by at least forming a self-assembled monolayer.

14. The method of claim 13 wherein the self-assembled monolayer comprises a thiol-terminated 6-mercaptohexanol self-assembled monolayer.

15. The method of claim 12 wherein the microfluidic blood plasma separator is removably clamped onto the substrate.

16. The method of claim 12 wherein the plasmonic array biosensor comprising a hole-disc array.

17. The method of claim 16 wherein the hole-disc array has period of 0.5-0.6 μm, a diameter of 0.1-0.3 μm, and a relief depth of 0.2-0.4 μm.

18. The method of claim 12 further comprising forming a metallic back reflector on the plasmonic array biosensor.

19. The method of claim 12 further comprising forming a dielectric polymer base for the plasmonic array biosensor.

20. The method of claim 19 further comprising forming a waterproof membrane on the dielectric polymer base.
